(19) United States
(12) Patent Application Publication
(10) Pub. No.: US 2025/0017959 A1
(43) Pub. Date: Jan. 16, 2025

(54) METHODS FOR TREATING OR PREVENTING VIRAL INFECTION

(71) Applicants: UNIKUM THERAPEUTICS APS, København N (DK); AARHUS UNIVERSITY, Aarhus (DK)

(72) Inventors: Martin Roelsgaard JAKOBSEN, Aarhus (DK); Renee VAN DER SLUIS, Aarhus (DK); Rasmus Otkjær BAK, København N (DK)

(21) Appl. No.: 18/708,110

(22) PCT Filed: Nov. 7, 2022
(86) PCT No.: PCT/EP2022/080987

§ 371 Date: May 7, 2024

(30) Foreign Application Priority Data
Nov. 8, 2021 (GB) 2116003.1

(51) Int. Cl. A61K 35/15; A61P 31/12; C12N 5/0784

(52) U.S. Cl. CPC A61K 35/15; A61P 31/12; C12N 5/0639; C12N 2506/11; C12N 2510/00

(57) ABSTRACT

The present invention relates to methods for treating or preventing viral infection, or disease or complications associated with viral infection, in particular infection by a coronavirus or influenza virus, optionally SARS-CoV-2, SARS-CoV or MERS-CoV.

### FIELD OF THE INVENTION

The present invention relates to methods for treating or preventing viral infection, or disease or complications associated with viral infection, in particular infection by a coronavirus or influenza virus, optionally SARS-CoV-2, SARS-CoV or MERS-CoV. The invention relates particularly to methods for treating or preventing COVID-19.

### BACKGROUND OF THE INVENTION

A severe viral acute respiratory syndrome named COVID-19 was first reported in Wuhan, China in December 2019. The virus rapidly disseminated globally leading to the pandemic with >70M confirmed infections and over 1.6M deaths in 12 months. The causative agent, SARS-CoV-2, is a beta coronavirus, related to SARS-CoV-1 and MERS coronaviruses. These viruses also cause severe inflammatory disease, typically with respiratory symptoms. Similar inflammatory and/or respiratory disease may be observed with infection by influenza and other viruses.

The impact of COVID-19 is variable between individuals and a great effort is made to understand why some people develop mild disease whilst others require hospitalization. A reported driver of severity is the imbalanced induction of an immune response consisting of a broad range of inflammatory cytokines (potentially leading to the excessive inflammatory response known as a ‘cytokine storm’) combined with a delayed induction of antiviral interferons (IFNs). Factors associated with severe COVID-19 include inborn errors in the toll-like receptor (TLR)3 and interferon regulatory factor (IRF)7-dependent type I IFN production and the presence of auto-antibodies against type I IFNs. This suggests that sufficient amounts of IFNs are essential for controlling the infection. Yet, it remains unclear which immune cells detect SARS-CoV-2 and initiate both the helpful anti-viral (IFN) and the unhelpful (excessive) inflammatory responses. Identifying the cellular sources of these responses is critical to develop targeted treatment and mitigate COVID-19 severity. There is a need for methods which promote an anti-viral response without undesirable (excessive) inflammatory response.

### SUMMARY OF THE INVENTION

Provided herein is a method of treating or preventing a disease in a subject comprising administering a plasmacytoid dendritic cell (pDC), or a composition comprising said cell, to the subject, wherein the disease is a viral infection or a disease or complication associated with a viral infection. The viral infection may be a respiratory viral infection. The virus may be a coronavirus or influenza virus. The virus may be SARS-CoV-2, SARS-CoV or MERS-CoV. The virus is preferably SARS-CoV-2. The disease is preferably COVID-19.

The method may additionally comprise administration of an anti-inflammatory agent to the subject. The pDC may be unable to express IL-6 or exhibit reduced IL-6 expression. The pDC may be engineered by transformation with an exogenous construct which prevents or reduces IL-6 expression. The pDC may exhibit reduced or no CD304 expression. The pDC may be engineered by transformation with an exogenous construct which deletes or disrupts CD304. CD304 is also known as Neuropilin-1, NPR1 and BDCA-4, and these terms are used interchangeably herein.

Also provided is a plasmacytoid dendritic cell, such as an engineered plasmacytoid dendritic cell as described herein, or a composition comprising said cell, which is optionally for use in a method of treating or preventing a viral infection or a disease or complication associated with a viral infection. The composition may be a pharmaceutical composition which additionally comprises a pharmaceutically acceptable diluent, excipient or carrier. The composition may also comprise an anti-inflammatory agent.

Also provided is use of a plasmacytoid dendritic cell, such as an engineered plasmacytoid dendritic cell as described herein, or a composition comprising said cell, in the manufacture of a medicament for treating or preventing a viral infection or a disease or complication associated with a viral infection.

Also provided is a method of generating a plasmacytoid dendritic cell, or a composition comprising said cell, the method comprising:
(a) providing hematopoietic stem progenitor cells (HSPCs);
(b) incubating said HSPCs in one or more media comprising cytokines, growth factor and/or interferons (IFNs), whereby said HSPCs are differentiated into precursor-pDCs and into pDCs incubating said HSPCs in one or more media, which media may comprise one or more cytokines, growth factors, interferons (IFNs) and/or aryl hydrocarbon receptor (1) antagonists (such as stemregenin-1), whereby said HSPCs are differentiated into precursor-pDCs and into pDCs;
(c) transforming said HSPCs prior to differentiation, or transforming said pDCs subsequent to differentiation, with an exogenous construct which prevents or reduces IL-6 expression and/or an exogenous construct which prevents or reduces CD304 expression which prevents or reduces CD304 expression; and optionally formulating the pDCs with a pharmaceutically acceptable diluent, excipient or carrier.

### BRIEF DESCRIPTION OF THE FIGURES

More detailed discussion of the Figures is provided in the Examples.

FIG. 1—shows evidence that plasmacytoid DCs can sense SARS-CoV-2 and induce an inflammatory response.

FIG. 2—shows evidence that induction of inflammatory cytokines enhances with increasing viral titers and duration of exposure.

FIG. 3—shows evidence that plasmacytoid DCs are not a reservoir for SARS-CoV-2 replication.

FIG. 4—shows evidence that cytokines secreted by plasmacytoid DCs after sensing of SARS-CoV-2, protect lung epithelial cells from de novo SARS-CoV-2 infection.

FIG. 5—shows evidence of the nature and timing of SARS-CoV-2-induced gene expression changes in pDCs.

FIG. 6—shows evidence of SARS-CoV-2-induced gene expression changes in pDCs.

FIG. 7—shows a Reactome Pathway Analysis for donor D^{high}.

FIG. 8—shows a Reactome Pathway Analysis for donor D^{low}.

FIG. 9—shows evidence that SARS-CoV-2 sensing and inflammatory cytokine induction by pDCs is mediated predominantly via MyD88.

FIG. 10—shows validation of MyD88^{KO} HSPC-pDCs.

FIG. 11—shows validation of TRIF^{KO} and RIG-I^{KO} HSPC-pDCs.

FIG. 12—shows evidence that SARS-CoV-2 sensing and subsequent IFNα production by pDCs is solely mediated by TLR7.

FIG. 13—shows validation of TLR3^{KO}, TLR7^{KO}, TLR8^{KO} and TRL7+TLR8^{KO} HSPC-pDCs.

FIG. 14—shows evidence that IL-6 production by pDCs is induced by TLR2/6-mediated sensing of the SARS-CoV-2 envelope protein.

FIG. 15—shows validation of TLR1^{KO}, TLR2^{KO} and TLR6^{KO} HSPC-pDCs.

FIG. 16—shows evidence that SARS-CoV-2 uses neuropilin-1 as immune evasion strategy by inhibiting type I IFNα production from pDCs.

### DETAILED DESCRIPTION OF THE INVENTION

Plasmacytoid Dendritic Cells (pDCs)

The invention relates to plasmacytoid dendritic cells (pDCs), which may preferably be stem cell-derived plasmacytoid dendritic cells.

In certain embodiments, pDCs are autologous. Autologous pDCs are advantageous for use in the prevention or treatment of disease in subjects because they minimise any risk of rejection of the transferred cells. In alternative embodiments, the cells are allogenic, such as isolated from healthy donors. Such treatments can potentially be prepared more quickly and offered “off the shelf”. In certain embodiments, the cells are or have been cryopreserved. Moreover, the cells may be xenogeneic.

The pDC may be unable to express IL-6 or exhibits reduced IL-6 expression, but which retains the ability to produce type I and III IFNs in response to virus. Such cells are useful in methods to treat or prevent disease. Expression of IL-6 by pDC may be prevented or reduced by any suitable method. For example, the plasmacytoid dendritic cell may be engineered to prevent or reduce IL-6 expression. In preferred embodiments, IL-6 expression by the pDC is prevented or reduced by deletion or disruption of TLR2. For example, the plasmacytoid dendritic cell may be engineered to prevent or reduce TLR2 expression or to delete or disrupt TLR2.

A reduction in IL-6 expression may be defined as a reduced quantity of IL-6 expression relative to the level of IL-6 expression from a suitable control cell when measured in the same assay. A suitable control cell may be a pDC obtained from the same source (e.g. differentiated from HSPC from the same subject), but against which no action to prevent or reduce IL-6 expression has been taken. For example, it may be a pDC which has not been engineered to prevent or reduce IL-6 expression. Quantification of IL-6 expression and/or determination of the IL-6 concentration level in a sample may be achieved by any suitable method. For example, IL-6 expression can be quantified by RT-qPCR. IL-6 expression and concentration level in a sample may be quantified by an ELISA assay. Suitable methods are also described in the Examples.

The pDC may exhibit reduced or no CD304 expression. Such cells are useful in methods to treat or prevent disease. The examples demonstrate that viruses such as SARS-CoV-2 impair type I IFNα production by pDCs by binding CD304. Expression of CD304 may be prevented or reduced by any suitable method. For example, the plasmacytoid dendritic cell may be engineered to prevent or reduce CD304 expression or to delete or disrupt CD304.

By engineered, it is typically meant that a plasmacytoid dendritic cell has been transformed by an exogenous construct. In some embodiments, the exogenous construct is a viral construct. In some embodiments, the viral construct is an AAV construct, an adenoviral construct, a lentiviral construct, or a retroviral construct.

In some embodiments, the exogenous construct is integrated into the genome of the engineered cell. In some embodiments, the exogenous construct is not integrated into the genome of the engineered cell. In some embodiments, the exogenous construct is introduced by a transposase, retrotransposase, episomal plasmid, mRNA, or random integration. Preferably, the exogenous construct is introduced with a gene editing system such as TALEN, zinc finger or, most preferably, CRISPR/Cas9.

The term exogenous as used herein has its normal meaning. In particular, the exogenous construct is a construct that has been introduced into the cell and that is not present in an unmodified cell in the same configuration or location. The exogenous construct may be constructed using endogenous (preferably human) sequences.

A preferred exogenous construct prevents IL-6 expression by the pDC. Particularly preferred is the use of CRISPR/Cas9 editing to remove or inactivate the IL-6 gene of a HSPC (optionally obtained from the subject) prior to or after differentiating it into a pDC in accordance with the methods described herein. The result is a pDC that does not express IL-6 but retains the ability to produce type I and III IFNs in response to virus. This particular approach and the use of this type of exogenous construct is discussed further in the Examples.

The person of skill in the art will be aware of other suitable approaches, and other suitable exogenous constructs, to provide a pDC which is unable to express IL-6 or exhibits reduced IL-6 expression.

An exogenous construct that prevents or reduces IL-6 expression by the pDC may be an exogenous construct that deletes or disrupts IL-6. Preferred is the use of CRISPR/Cas9 editing to remove or inactivate the IL-6 gene of a HSPC (optionally obtained from the subject) prior to or after differentiating it into a pDC in accordance with the methods described herein. The result is a pDC that does not comprise active IL-6 and does not express IL-6 but retains the ability to produce type I and III IFNs in response to virus, as demonstrated in the Examples. This particular approach and the use of this type of exogenous construct is discussed further in the Examples.

A preferred exogenous construct that prevents or reduces IL-6 expression by the pDC is an exogenous construct that deletes or disrupts TLR2. Particularly preferred is the use of CRISPR/Cas9 editing to remove or inactivate the TLR2 gene of a HSPC (optionally obtained from the subject) prior to or after differentiating it into a pDC in accordance with the methods described herein. The result is a pDC that does not comprise active TLR2 and does not express IL-6 but retains the ability to produce type I and III IFNs in response to virus, as demonstrated in the Examples. This particular approach and the use of this type of exogenous construct is discussed further in the Examples.

Accordingly, preferred pDCs for use in the invention comprise deletion or disruption of TLR2. Deletion may be partial or complete.

Further preferred exogenous constructs for use in the invention are exogenous constructs that prevent or reduce CD304 expression, for example by deleting or disrupting CD304. Particularly preferred is the use of CRISPR/Cas9 editing to remove or inactivate the CD304 gene of a HSPC (optionally obtained from the subject) prior to or after differentiating it into a pDC in accordance with the methods described herein. The result is a pDC that does not comprise active CD304 and retains the ability to produce type I and III IFNs in response to virus, as demonstrated in the Examples. This particular approach and the use of this type of exogenous construct is discussed further in the Examples.

Accordingly, preferred pDCs for use in the invention comprise deletion or disruption of CD304. Deletion may be partial or complete.

In preferred embodiments, a pDC for use in the invention comprises deletion or disruption of both TLR2 and CD304.

The person of skill in the art will be aware of appropriate methods and constructs useful for deleting or disrupting genes such as IL-6, TLR2 and CD304 in accordance with the invention. The examples provide exemplary guide RNAs that may be used, for example with CRISPR/Cas9 editing. The person of skill in the art is able to design appropriate guide RNAs, for example using the guidance in the Examples. The person of skill in the art will also be aware of alternative approaches, including using alternative nucleases such as meganuclease, a zinc finger nuclease (ZFN), a transcription-activator like effector nuclease (TALEN), a homing endonuclease, a CRISPR-associated endonuclease or a modified CRISPR-associated endonuclease.

Methods for Generating pDCs

The pDCs may be generated by any appropriate method. Exemplary methods for generating pDCs in significant amounts are provided in WO2018/206577. The invention also provide methods of generating engineered plasmacytoid dendritic cells.

In certain embodiments, the method for producing plasmacytoid dendritic cell (pDCs) comprises:
providing hematopoietic stem progenitor cells (HSPCs)
incubating said HSPCs in one or more media, which media may typically comprise one or more cytokines, growth factors, interferons (IFNs) and/or aryl hydrocarbon receptor (1) antagonists (such as stemregenin-1), whereby said HSPCs are differentiated into precursor-pDCs and into pDCs.

In certain embodiments, the method for producing plasmacytoid dendritic cell (pDCs) comprises:
providing hematopoietic stem progenitor cells (HSPCs),
incubating said HSPCs in one or more media, which media may typically comprise one or more cytokines, growth factors, interferons (IFNs) and/or aryl hydrocarbon receptor (1) antagonists (such as stemregenin-1), whereby said HSPCs are differentiated into precursor-pDCs and into pDCs,
transforming said pDCs with an exogenous construct which prevents or reduces IL-6 expression and/or an exogenous construct which prevents or reduces CD304 expression.

Accordingly, in certain embodiments, the method for producing an engineered plasmacytoid dendritic cell (pDCs) comprises:
providing hematopoietic stem progenitor cells (HSPCs),
incubating said HSPCs in one or more media, which media may typically comprise one or more cytokines, growth factors, interferons (IFNs) and/or aryl hydrocarbon receptor (1) antagonists (such as stemregenin-1), whereby said HSPCs are differentiated into precursor-pDCs and into pDCs, and
transforming said HSPCs prior to differentiation, or transforming said pDCs subsequent to differentiation, with an exogenous construct which prevents or reduces IL-6 expression and/or an exogenous construct which prevents or reduces CD304 expression;

Transforming the cells can be achieved by any appropriate technique.

In preferred embodiments, the exogenous construct which prevents or reduces IL-6 expression is an exogenous construct that deletes or disrupts TLR2.

In preferred embodiments, the exogenous construct which prevents or reduces CD304 expression is an exogenous construct that deletes or disrupts CD304.

In further preferred embodiments, the method comprises transforming said HSPCs prior to differentiation, or transforming said pDCs subsequent to differentiation, with an exogenous construct that deletes or disrupts TLR2 and an exogenous construct that deletes or disrupts CD304.

In some embodiments the exogenous construct is a viral construct. In some embodiments, the viral construct is an AAV construct, an adenoviral construct, a lentiviral construct, or a retroviral construct. The construct may comprise a reporter gene such as GFP, mCherry, truncated EGFR, or truncated tNGFR to aid sorting of pDCs with the construct.

In preferred embodiments, CD34⁺ HSPC are transformed and then differentitaed into pDCs.

In certain embodiments, the method for producing plasmacytoid dendritic cell (pDCs) comprises:
providing hematopoietic stem progenitor cells (HSPCs)
transforming said HSPCs with an exogenous construct comprising an extracellular antigen recognition domain that recognises an antigen on a target cell,
incubating said HSPCs in a first medium comprising cytokines and growth factor whereby said HSPCs are differentiated into precursor-pDCs
adding interferons (IFNs) to said first medium to obtain a second medium whereby said precursor-pDCs are differentiated into pDCs

In certain embodiments, the method for producing plasmacytoid dendritic cell (pDCs) comprises:
providing hematopoietic stem progenitor cells (HSPCs)
transforming said HSPCs with an exogenous construct which prevents or reduces IL-6 expression and/or an exogenous construct which prevents or reduces CD304 expression;
incubating said HSPCs in a first medium comprising cytokines and growth factor whereby said HSPCs are differentiated into precursor-pDCs
adding stem cell factor (SCF) and StemRegnin 1 (SR1) in a first medium to obtain high yield of pre-cursor pDCs
providing a second medium comprising interferons (IFNs)
adding interferons (IFNs) to said a second medium to said first medium comprising pre-cursor pDCs, whereby said precursor-pDCs are transformed into to obtain a high yield of fully activated and differentiated pDCs a second medium
whereby said precursor-pDCs are differentiated into pDCs

In preferred embodiments said second medium comprises IFN-γ and/or IFN-β. In another embodiment said second medium further comprises IL-3. Preferably, said second medium comprises IL-3, IFN-γ and IFN-β.

The precursor-pDCs may for example be incubated for at least 24 hours in said second medium. Preferably, said precursor-pDCs are incubated for 24 to 72 hours in said second medium.

In one embodiment said first medium comprises Flt3 ligand, thrombopoietin and/or interleukin-3. In another embodiment said first medium further comprises stem cell factor and StemRegenin 1. In another embodiment said first medium further comprises stem cell factor and UM 171. In another embodiment said first medium further comprises RPMI medium supplemented with fetal calf serum (FCS). In another embodiment said first medium comprises serum-free medium (SFEM). Preferably, said first medium comprises Flt3 ligand, thrombopoietin, SCF, interleukin-3 and StemRegenin 1.

The HSPCs may for example be incubated for 21 days in said first medium. In one embodiment the method as described herein, further comprises a step of immunomagnetic negative selection to enrich for differentiated pDCs.

“Hematopoietic stem cells” (HSCs) as used herein are multipotent stem cells that are capable of giving rise to all blood cell types including myeloid lineages and lymphoid lineages. Myeloid lineages may for example include monocytes and macrophages, neutrophils, basophils, eosinophils, erythrocytes, megakaryocytes/platelets and dendritic cells, whereas lymphoid lineages may include T-cells, B-cells and NK-cells.

In a preferred embodiment HSCs are Hematopoietic stem and progenitor cells (HSPCs). HSCs or HSPCs are found in the bone marrow of humans, such as in the pelvis, femur, and sternum. They are also found in umbilical cord blood and in peripheral blood.

Stem and progenitor cells can be taken from the pelvis, at the iliac crest, using a needle and syringe. The cells can be removed as liquid for example to perform a smear to look at the cell morphology or they can be removed via a core biopsy for example to maintain the architecture or relationship of the cells to each other and to the bone.

The HSCs or HSPCs may also be harvested from peripheral blood. To harvest HSCs or HSPCs from the circulating peripheral blood, blood donors can be injected with a cytokine that induces cells to leave the bone marrow and circulate in the blood vessels. The cytokine may for example be selected from the group consisting of granulocyte-colony stimulating factor (G-CSF), GM-CSF granulocyte-macrophage colony-stimulating factor (GM-CSF) and cyclophosphamide. They are usually given as an injection into the fatty tissue under the skin every day for about 4-6 days.

The HSCs or HSPCs may also be harvested or purified from bone marrow. Stem cells are 10-100 times more concentrated in bone marrow than in peripheral blood. The hip (pelvic) bone contains the largest amount of active marrow in the body and large numbers of stem cells. Harvesting stem cells from the bone marrow is usually done in the operating room.

HSCs or HSPCs may also be purified from human umbilical cord blood (UCB). In this method, blood is collected from the umbilical cord shortly after a baby is born. The volume of stem cells collected per donation is quite small, so these cells are usually used for children or small adults.

The first medium is a differentiation medium, wherein HSCs are differentiated into precursor-pDCs. Thus, the first medium comprises differentiation factors.

Before differentiation of HSCs into precursor-pDCs, the HSCs may be cultured in a culture medium not comprising differentiation factors. The culture medium may be supplemented with conventional cell culture components such as serum, such as for example fetal calf serum, b-mercaptoethanol, antibiotics, such as penicillin and/or streptomycin, nutrients, and/or nonessential amino acids. Conventional cell culture components can also be substituted for conventional serum-free medium supplemented with conventional penicillin and/or streptomycin.

To initiate differentiation of HSCs into precursor-pDCs, differentiation factors, such as Flt3 ligand, thrombopoietin and/or at least one interleukin selected from interleukin-3, IFN-b and PGE2 are added to the medium. SCF and/or SR1 can also be used.

Thus, in a preferred embodiment said first medium comprises Flt3 ligand, thrombopoietin and/or at least one interleukin selected from interleukin-3, IFN-b and PGE2. More preferably, said first medium comprises Flt3 ligand, thrombopoietin and/or interleukin-3. In another preferred embodiment, the first medium comprises SCF and/or SR1

Appropriate culture media can be prepared by the skilled person, for example using the guidance in WO 2018/206577.

The HSPCs are incubated in the first medium under conditions that are typical for human cell cultures and well known to the skilled person. Typical conditions for incubation of cell cultures are for example a temperature of 37° C., 95% humidity and 5% CO₂.

In one embodiment the HSPCs are incubated for at least 1 day, such as at least 2 days, at least 3 days, such as for example at least 4 days, such as at least 5 days, at least 6 days, such as for example at least 7 days, such as at least 8 days, at least 9 days, such as for example at least 10 days, such as at least 12 days, at least 14 days in said first medium. In a more preferred embodiment the culture is incubated for at least 16 days, such as at least 18 days, at least 20 days or such as for example at least 21 days in said first medium.

The HSCs may for example be incubated for 1 week, 2 weeks, 3 weeks or 4 weeks in said first medium. In a preferred embodiment said HSPCs are incubated for 21 days in said first medium.

In one embodiment the first medium is refreshed during the incubation period. The medium may for example be refreshed every second day, every third day or every fourth day during the incubation period. The first medium is preferably refreshed with medium containing one or more components of the first medium as described herein and above.

Preferably the medium is refreshed with medium comprising the cytokines.

After incubation of HSPCs in the first medium, wherein HSCs are differentiated into precursor-pDCs, IFNs are added to the first medium thereby obtaining a second medium.

Alternatively, a second medium is provided, which comprises IFNs, such as IFN type I, IFN type II and/or IFN type III.

In one embodiment said second medium comprises IFN-α, IFN-γ and/or IFN-β.

In one preferred embodiment said second medium comprises IFN-γ and/or IFN-β. Preferably, said second medium comprises IFN-γ and IFN-β.

In another preferred embodiment said second medium comprises interleukin-3 (IL-3).

In the embodiment, wherein the first medium comprises IL-3, IL-3 may be added to the medium again, for example together with the interferons. It is understood that the three components can be added in any order. In a particular preferred embodiment said second medium comprises IFN-γ, IFN-β and IL-3.

The precursor-pDCs are incubated in the second medium under conditions that are typical for human cell cultures and well known to the skilled person. Typical conditions for incubation of cell cultures are for example a temperature of 37° C., 95% humidity and 5% CO₂.

In one embodiment said precursor-pDCs are incubated in said second medium for at least 1 hour, such as at least 5 hours, such as for example at least 10 hours, such as at least 15 hours or such as at least 20 hours in said second medium. In one preferred embodiment precursor-pDCs are incubated for at least 24 hours in said second medium.

In another embodiment said precursor-pDCs are incubated in said second medium for at least 1 day, at least two days, at least three days or at least 4 days.

Any of the methods described above may further comprise formulating the pDC or a population of said pDCs in a composition, for example by formulating the pDC with a pharmaceutically acceptable preservative, diluent, excipient, or carrier. Suitable compositions, preservatives, diluents, excipients, and carriers are discussed in more detail in a separate section.
Methods for Treating or Preventing Viral Infection, or a Disease or Complication Associated with a Viral Infection

The present invention is concerned with treating or preventing viral infections, and/or diseases or complications associated with viral infection. The diseases or complications associated with viral infection are typically severe inflammatory reactions (such as cytokine storm) and/or acute organ damage resulting from such reactions. The present invention is thus particularly concerned with viral infections that are associated with such severe inflammatory responses. Other diseases or complications associated with viral infection are long term or chronic in nature. For example, a complication associated with viral infection may be asthma. The present invention is thus particularly concerned with viral infections that are associated with long term or chronic complications, in particular asthma.

Such viral infections may include a coronavirus infection or an influenza infection. The coronavirus may be severe acute respiratory syndrome coronavirus 2 (SARS-CoV-2), severe acute respiratory syndrome coronavirus 1 (SARS-CoV-1), Middle East respiratory syndrome-related coronavirus (MERS-CoV), or any related or similar virus. The patient may not (yet) exhibit overt symptoms of viral infection, but will typically exhibit one or more symptoms of a disease associated with viral infection, particularly symptoms affecting the respiratory system. The patient may be exhibiting one or more symptoms of the coronavirus disease COVID-19. Symptoms may include fever, cough, shortness of breath or difficulty breathing, loss of smell and/or taste, tiredness, aches, runny nose, sore throat. Confirmation of a viral infection may be made by any suitable assay. For example, a real-time reverse-transcription polymerase chain reaction (rRT-PCR) assay may be used to detect viral RNA in a clinical sample from the patient. Treatment is preferably administered to the patient prior to any respiratory symptom becoming severe. Patients who are particularly likely to develop severe symptoms are older people, people with suppressed immunity, and those with underlying medical problems such as cardiovascular disease, diabetes, chronic respiratory disease, and cancer. Treatment is particularly suitable for such patients.

The viral infection may be a respiratory viral infection.

In terms of the cells of the immune system which can detect and respond to viral infection, alveolar macrophages seem incapable of sensing SARS-CoV-2, whereas lung epithelial cells detect the virus and produce type I IFNβ and type III IFNλ1, but only after initiation of virus replication. Plasmacytoid dendritic cells (pDCs), however, are an autonomous cell type and major producers of type I IFNα, making them potentially pivotal for the human immune system to control viral infections. Clinical studies reveal that severe COVID-19 cases have a reduction in circulating pDCs as well as minimal influx of pDCs into the lungs compared to patients with moderate disease and healthy controls. These severe cases of COVID-19 also exhibit reduced type I IFNα, type III IFNλ and Interleukin(IL-)3 levels in plasma, of which IL-3 is known to be important for pDC function. Whether disease severity is due to the lack of pDCs in the lungs, dysfunctional immunological activity of pDCs, or that the pDCs' potential anti-viral mechanism contributes to disease development remains unclear. Since pDCs also secrete inflammatory cytokines such as IL-6, which can contribute to undesirable (excessive) inflammatory responses such as cytokine storm, this may also contribute.

The inventors have determined that pDCs sense SARS-CoV-2 and in response produce both multiple inflammatory (IL-6, IL-8, CXCL10) and anti-viral (type I IFNα and type III IFNλ1) cytokines. Importantly, the cytokine response elicited by pDCs is sufficient to protect epithelial cells from de novo SARS-CoV-2 infection. The inventors have identified MyD88 as the main adaptor molecule responsible for the response, but surprisingly also identified a unique time-dependent and clear biphasic expression pattern of genes, independently of virus replication, suggesting a multifaceted sensory mechanism of SARS-CoV-2 in pDCs. In line with this, initiation of IL-6 production in response to the virus occurred independently of MyD88, though the antiviral type I and III IFNs was solely dependent on MyD88. As such, the desirable anti-viral responses of pDCs can be separated from the less desirable inflammatory responses, and thus prevention or treatment of COVID-19 (and similar viral diseases) may be achieved by suitable administration of pDCs.

The pDC (or a composition thereof) may be used in therapy or prophylaxis. In therapeutic applications, pDC or compositions are administered to a subject already suffering from a disorder or condition, in an amount sufficient to cure, alleviate or partially arrest the condition or one or more of its symptoms. Such therapeutic treatment may result in a decrease in severity of disease symptoms, or an increase in frequency or duration of symptom-free periods. An amount adequate to accomplish this is defined as “therapeutically effective amount”. In prophylactic applications, pDC or compositions are administered to a subject not yet exhibiting symptoms of a disorder or condition, in an amount sufficient to prevent or delay the development of symptoms. Such an amount is defined as a “prophylactically effective amount”. The subject may have been identified as being at risk of developing the disease or condition by any suitable means. Thus the invention also provides a pDC or composition thereof for use in the treatment of the human or animal body. Also provided herein is a method of prevention or treatment of disease or condition in a subject, which method comprises administering a pDC or composition to the subject in a prophylactically or therapeutically effective amount.

The pDC (or a composition thereof) may reduce viral replication in a subject. Viral replication may be determined via any suitable method. Suitable methods are discussed in the Examples.

The invention provides a method of treating or preventing a disease in a subject comprising administering a pDC (or a composition thereof) to a subject. Therefore, the invention provides a new adoptive cell therapy. Adoptive cell therapy is the transfer of ex vivo grown cells, most commonly immune-derived cells, into a host with the goal of transferring the immunologic functionality and characteristics of the transferred cells. Adoptive cell therapy is well established for treating cancer and autoimmune and inflammatory diseases, albeit using different transferred immune cells with different immune-regulating effects and activities.

In certain embodiments of the invention, the methods of treatment may comprise (i) collecting autologous hematopoietic stem progenitor cells (HSPCs), either from the subject to be treated or a healthy donor; (ii) preparing pDCs or engineered pDCs, for example using a method discussed herein; (iii) optionally administering to the subject lymphodepleting chemotherapy; and (iv) administering to the subject the pDCs or engineered pDCs.

The methods of the invention may comprise administering pDC (or a composition thereof) which are engineered by transformation with one or more exogenous construct. Individual cells may express more than one construct, or the population of cells administered may comprise a plurality of different cells expressing different constructs.

In certain embodiments, the cells may be isolated from a subject and used fresh, or frozen for later use, in conjunction with (e.g., before, simultaneously or following lymphodepletion.

In certain embodiments, the cells may be administered to the subject by dose fractionation, wherein a first percentage of a total dose is administered on a first day of treatment, a second percentage of the total dose is administered on a subsequent day of treatment, and optionally, a third percentage of the total dose is administered on a yet subsequent day of treatment.

An exemplary total dose comprises 10³ to 10¹¹ cells/kg body weight of the subject, such as 10³ to 10¹⁰ cells/kg body weight, or 10³ to 10⁹ cells/kg body weight of the subject, or 10³ to 10⁸ cells/kg body weight of the subject, or 10³ to 10⁷ cells/kg body weight of the subject, or 10³ to 10⁶ cells/kg body weight of the subject, or 10³ to 10⁵ cells/kg body weight of the subject. Moreover, an exemplary total dose comprises 10⁴ to 10¹¹ cells/kg body weight of the subject, such as 10⁵ to 10¹¹ cells/kg body weight, or 10⁶ to 10¹¹ cells/kg body weight of the subject, or 10⁷ to 10¹¹ cells/kg body weight of the subject.

An exemplary total dose may be administered based on a patient body surface area rather than the body weight. As such, the total dose may include 10³ to 10¹³ cells per m2.

In certain embodiments of the invention, the methods comprise lymphodepletion. Lymphodepletion may be achieved by any appropriate means. Lymphodepletion may be performed prior to administration of the engineered cells, or subsequent to. In certain embodiments, lymphodepletion is performed both before and after administration of the engineered cells.

In the therapeutic methods of the invention, cells are administered to a subject already suffering from viral infection, or a disease or complication associated with viral infection, in an amount sufficient to cure, alleviate or reduce the frequency of one or more symptoms. An amount adequate to accomplish this is defined as a “therapeutically effective amount”. The subject may have been identified as suffering from said viral infection, or said disease or complication, and being suitable for an adoptive cell transfer immunotherapy by any suitable means. In preventative methods of the method, cells are administered to a subject that may not have a confirmed viral infection, but may have a suspected infection or be considered at risk of such infection, in an amount sufficient to prevent or reduce the development of one or more symptoms. An amount adequate to accomplish this is defined as a “prophylactically effective amount”.

The method may comprise administration of an additional active ingredient, which may be an anti-viral agent, such as remdesivir, and/or an anti-inflammatory agent. The additional active ingredient may be administered simultaneously, concurrently, separately or sequentially with the pDC or composition thereof. The additional active ingredient may be present in the same composition as the pDC.

The anti-inflammatory agent is administered such that the desirable anti-viral effects of the pDCs (such as secretion of IFN type I and/or IFN type III cytokines) are retained but undesirable (excessive) inflammatory response(s) are controlled or suppressed. The anti-inflammatory agent may be administered simultaneously with the pDC, or at a suitable time interval subsequent to administration of the pDC. The time interval may be at least around 4 hours, 8 hours, 12 hours, or 24 hours. The separation in time permits the pDC to exert anti-viral effects and the anti-inflammatory agent reduces or suppresses other undesirable (excessive) inflammatory response(s).

The anti-inflammatory agent may have general activity, for example it may be a corticosteroid, such as dexamethasone, hydrocortisone or methylprednisolone; or a non-steroidal anti-inflammatory drug (NSAID), such as ibuprofen.

The anti-inflammatory agent may be more targeted, for example it may be an IL-6 antagonist; optionally comprising an antibody specific for IL-6 or IL-6R, such as tocilizumab, sarilumab, siltuximab, sirukumab, clazakizumab, elsilimomab, or levilimab. The anti-inflammatory agent may be a TLR2 antagonist. The anti-inflammatory agent may not be a TLR7 antagonist.

A general anti-inflammatory is preferably administered a suitable time interval after the pDC as described above. A targeted anti-inflammatory, such as an IL-6 antagonist, may be administered simultaneously with the pDC, but may also be administered separately such as after a suitable time interval as described above.

The pDC may be unable to express IL-6 or exhibit reduced IL-6 expression, as described in the preceding sections. For example, the pDC may comprise deletion or disruption of TLR2. The pDC may alternatively or additionally exhibit reduced or no CD304 expression, as described in the preceding sections. For example, the pDC may comprise deletion or disruption CD304. Accordingly, the pDC may comprise deletion or disruption of both TLR2 and CD304.

### Compositions

The invention also provides a composition comprising a pDC as defined herein, or a population of said pDC. The composition may be for use in a method of treating or preventing a viral infection or a disease or complication associated with a viral infection. The composition may be a pharmaceutical composition, and as such may comprise a pharmaceutically acceptable preservative, diluent, excipient, or carrier. The composition may comprise an additional active ingredient, which may be an anti-viral agent, such as remdesivir, and/or an anti-inflammatory agent. The anti-inflammatory agent may have general activity, for example it may be a corticosteroid, such as dexamethasone, hydrocortisone or methylprednisolone; or a non-steroidal anti-inflammatory drug (NSAID), such as ibuprofen.

The anti-inflammatory agent may be more targeted, for example it may be an IL-6 antagonist; optionally comprising an antibody specific for IL-6 or IL-6R, such as tocilizumab, sarilumab, siltuximab, sirukumab, clazakizumab, elsilimomab, or levilimab.

A general anti-inflammatory is preferably administered a suitable time interval after the pDC as described above. A targeted anti-inflammatory, such as an IL-6 antagonist, may be administered simultaneously with the pDC, but may also be administered separately such as after a suitable time interval as described above.

The various compositions of the invention may be formulated using any suitable method. Formulation of cells with standard pharmaceutically acceptable preservatives, diluents, carriers and/or excipients may be carried out using routine methods in the pharmaceutical art. The exact nature of a formulation will depend upon several factors including the cells to be administered and the desired route of administration. Suitable types of formulation are fully described in Remington's Pharmaceutical Sciences, 19th Edition, Mack Publishing Company, Eastern Pennsylvania, USA. The cells may be formulated so they may be administered by any route. Suitable routes include, but are not limited to, intravenous, intramuscular, subcutaneous, intraperitoneal, or intra-pulmonary route. The cells may also be administered directly to a tissue of interest, such as liver, kidney or lung tissue. The cells may be administered directly into a site of viral infection.

Compositions may be prepared together with a physiologically acceptable preservative, diluent, carrier or excipient. Typically, such compositions are prepared as liquid suspensions of cells. The cells may be mixed with an excipient which is pharmaceutically acceptable and compatible with the active ingredient. Suitable excipients are, for example, water, saline, dextrose, glycerol, of the like and combinations thereof. In addition, if desired, the pharmaceutical compositions of the invention may contain minor amounts of auxiliary substances such as wetting or emulsifying agents, pH buffering agents, and/or adjuvants which enhance effectiveness.

### General

It is to be understood that different applications of the disclosed products and methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, reference to “a polypeptide” includes “polypeptides”, and the like.

Unless specifically prohibited, the steps of a method disclosed herein may be performed in any appropriate order and the order in which the steps are listed should not be considered limiting.

All publications, patents and patent applications cited herein are hereby incorporated by reference in their entirety.

### Example 1

The severe acute respiratory syndrome coronavirus-2 (SARS-CoV-2) has, since its first appearance in 2019, resulted in a devastating pandemic of coronavirus disease 2019 (COVID-19) that prevails mid 2021 (3, 4). The severity of COVID-19 is highly variable between individuals and a great effort is made to understand why some people develop mild disease whilst others require hospitalization (5, 6). A reported driver of disease severity is the imbalanced induction of an immune response consisting of a broad range of inflammatory cytokines combined with a delayed induction of antiviral interferons (IFNs) (7-9). Factors associated with severe disease are inborn errors in the toll-like receptor (TLR)3 and interferon regulatory factor (IRF)7-dependent type I IFN production and the presence of auto-antibodies against type I IFNs (1, 10). This indicates that sufficient amounts of IFNs are essential for controlling the infection. Yet, it remains unclear which immune cells detect SARS-CoV-2 and initiate the inflammatory response. Alveolar macrophages seem incapable of sensing SARS-CoV-2 (11). Similarly, in vitro generated macrophages and classical myeloid DCs are unable to elicit the production of pro-inflammatory and anti-viral cytokines in response to SARS-CoV-2 (12). Lung epithelial cells, however, can detect the virus and produce type I IFNβ and type III IFNλ1, but only after initiation of virus replication (13, 14). Plasmacytoid dendritic cells (pDCs), however, are an autonomous cell type and major producers of type I IFNα, making them pivotal for the human immune system to control viral infections (15). Clinical studies reveal that severe COVID-19 cases have a reduction in circulating pDCs as well as minimal influx of pDCs into the lungs compared to patients with moderate disease and healthy controls (8, 16-18). These severe cases of COVID-19 also exhibited reduced type I IFNα, type III IFNλ and Interleukin (IL-)3 levels in plasma, of which IL-3 is known to be important for pDC function (18). Whether disease severity is due to the lack of pDCs in the lungs or due to dysfunctional cytokine production by the pDCs, remains unclear. The cytokine production from pDCs is triggered upon the innate detection of viral components via various extra- and intra-cellular receptors also known as pattern recognition receptors (PRRs). In particular, the Toll-like receptors (TLRs) and retinoic acid-inducible gene I (RIG-I)-like receptors (RLR) are the major receptor classes responsible for sensing RNA virus infection and triggering antiviral IFN production (19). In the present study, we explored via which molecular mechanism human pDCs can sense SARS-CoV-2, by using CRISPR-editing to screen for several innate immune sensor pathways that are required for the production of antiviral IFNs and inflammatory cytokines upon viral sensing.

Human pDCs Sense SARS-CoV-2 but are Refractory to Infection.

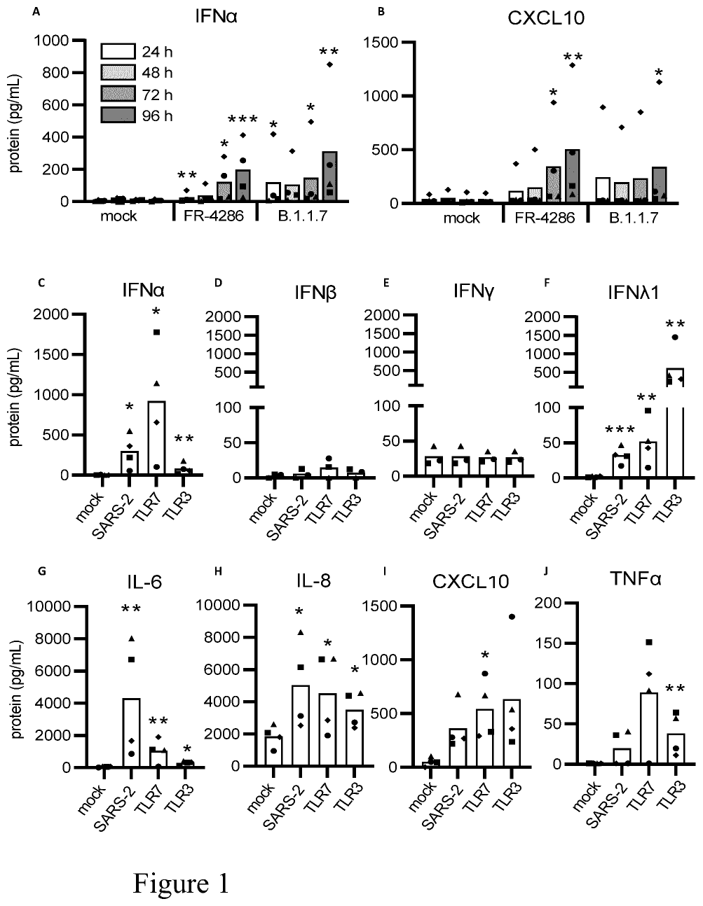

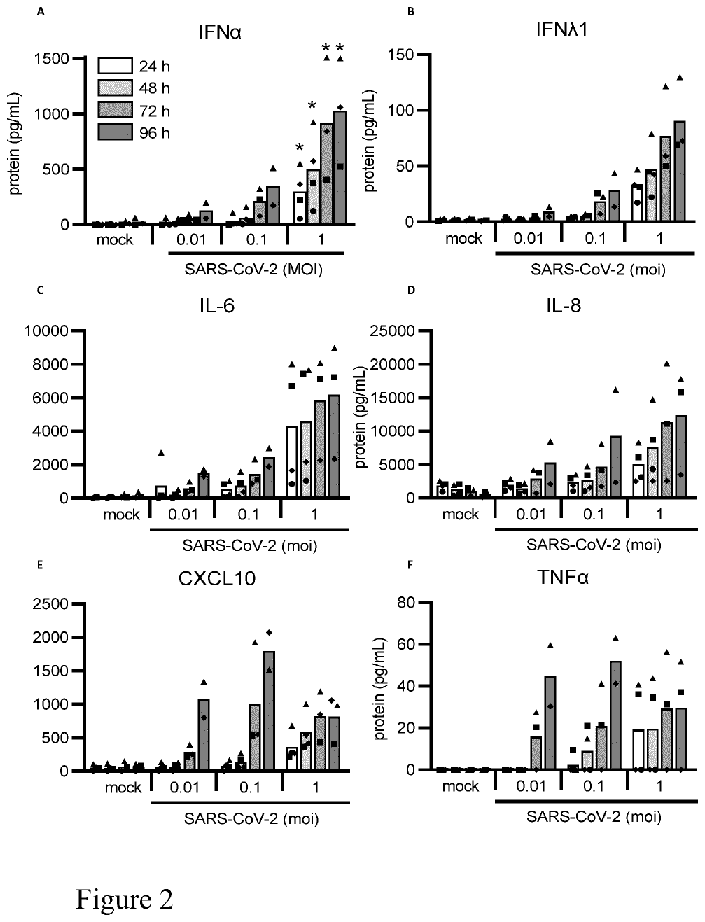

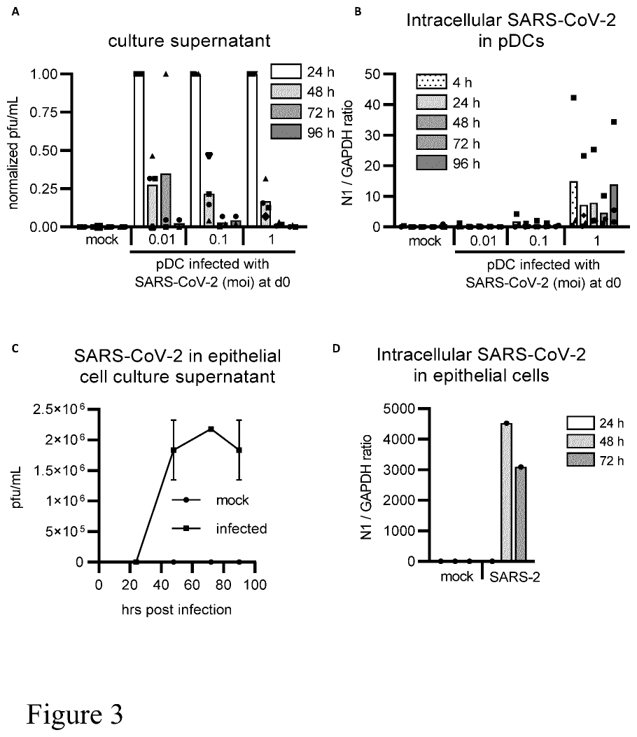

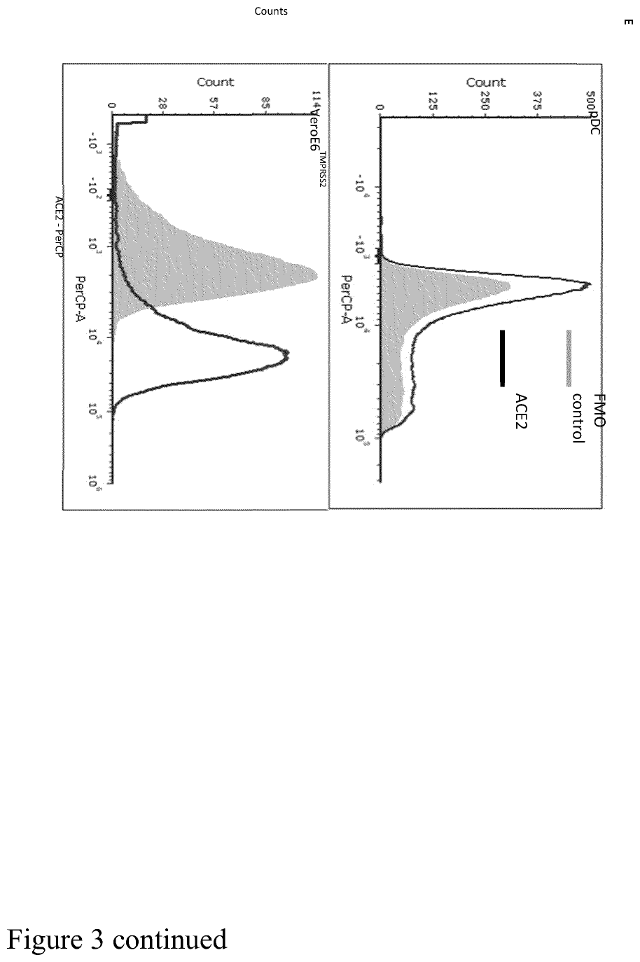

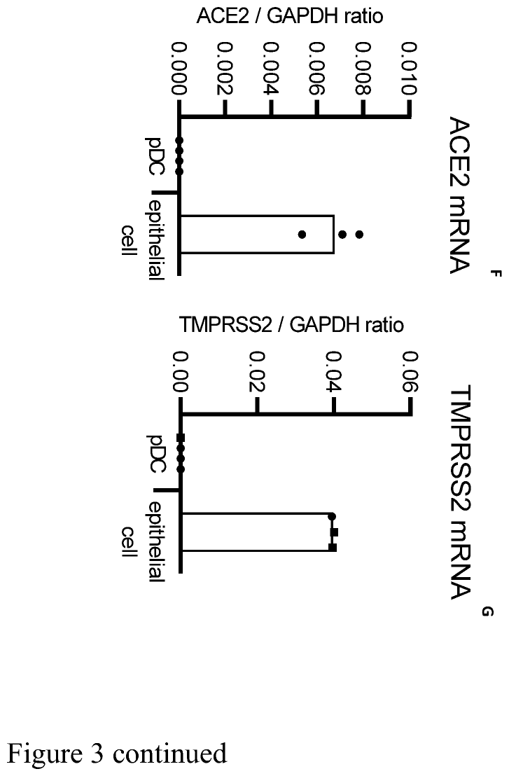

Studying viral sensing by human pDCs is hampered by the limited amount of pDCs that can be obtained from peripheral blood and their incapability to be genetically modified. To overcome this and enable the investigation of potential pDC sensing mechanisms of SARS-CoV-2, we adopted a cellular platform designed to generate human primary pDCs ex vivo using hematopoietic stem and progenitor cells (HSPC) from healthy individuals (20). The HSPC-derived pDCs, produced from different donors, were exposed to two different SARS-CoV-2 isolates; the Freiburg isolate (FR2020) which is an early 2020 Wuhan-like strain and the SARS-CoV-2 alpha variant. Type I IFNα and CXCL10 production was assessed longitudinally and found to be induced by both variants (FIG. 1A-B) with a trend towards a more rapid type I IFNα induction observed for the B.1.1.7 variant. To further characterize how pDCs sense SARS-CoV-2, we continued with the reference strain. First, pDCs were exposed to TLR agonists or SARS-CoV-2 and after 24 hrs, cell culture supernatants and pDCs were collected to assess a broader range of inflammatory cytokines at both the protein and mRNA levels. Following SARS-CoV-2 exposure, we observed increased production of type I IFNα and type III IFNλ1, but not type I IFNβ and type II IFNγ (FIG. 1C-F), resembling a TLR7 agonist response. IL-6, IL-8 and CXCL10 were likewise induced, with SARS-CoV-2 inducing higher IL-6 levels than the TLR7 ligand (FIG. 1G-I). Tumor necrosis factor (TNF)α was only marginally induced in pDCs from some donors challenged with SARS-CoV-2 (FIG. 1J), a pattern resembling neither TLR3 nor TLR7 agonists. Next, we evaluated the type I IFNα expression pattern relative to viral titer and duration of exposure. A viral MOI of 1 resulted in a strong type I IFNα response on both RNA and protein levels (FIG. 1K-L), and a clear positive correlation between type I IFNα induction and exposure time was observed (FIG. 1M). A similar pattern was observed for type III IFNλ1 and multiple inflammatory cytokines (FIG. 2). Next, we assessed whether SARS-CoV-2 was able to replicate in pDCs. However, no viral products indicative of SARS-CoV-2 replication were detected in pDCs (FIG. 3), which is supported by findings of others (21). Overall, these results demonstrate that pDCs are capable of sensing SARS-CoV-2 and in response produce type I IFNα and numerous inflammatory cytokines that are important to the cytokine storm observed in people suffering from severe COVID-19 disease (7-9).

Detection of SARS-CoV-2 by pDCs Facilitates a Protective Antiviral Response.

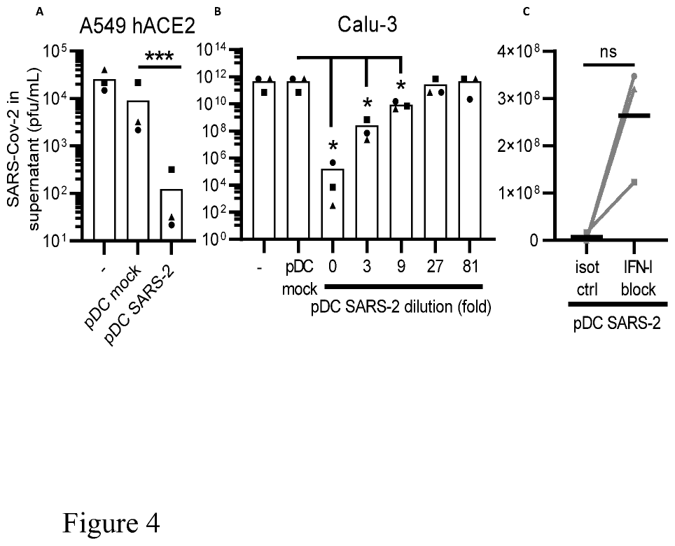

A hallmark of antiviral activity is protection of target cells against the pathogen. To investigate if pDC-secreted cytokines protected cells from SARS-CoV-2 infection, we next exploited two different lung epithelial cell types—A549 hACE2 and Calu-3—and exposed them to cell culture supernatant from pDCs that were either cultured as normal or exposed to SARS-CoV-2, followed by virus inoculation. Pre-treatment with supernatant from SARS-CoV-2-exposed pDCs reduced virus replication in both cell lines in a dose dependent manner (FIG. 4A-B). Using blocking antibodies, we found that this protection was mediated partially by type I IFNs (FIG. 4C). Overall, these data indicate that cytokines produced by pDCs in response to SARS-CoV-2 can protect lung cells from infection by reducing virus replication and thereby limit viral spread.

Detection of SARS-CoV-2 in pDCs Generates a Biphasic Time-Dependent Inflammatory Signature.

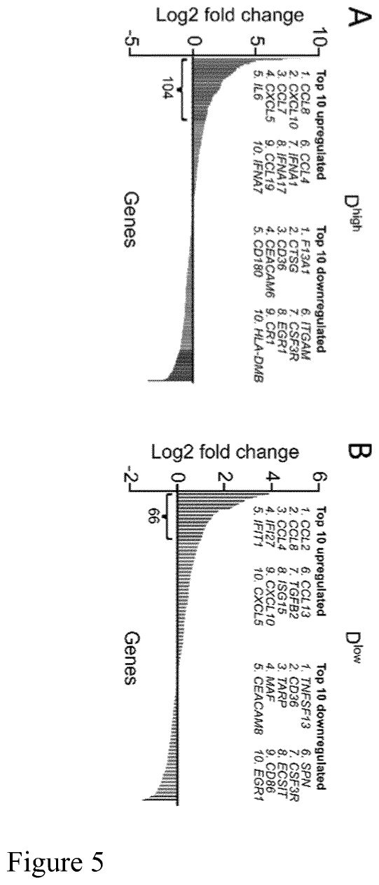

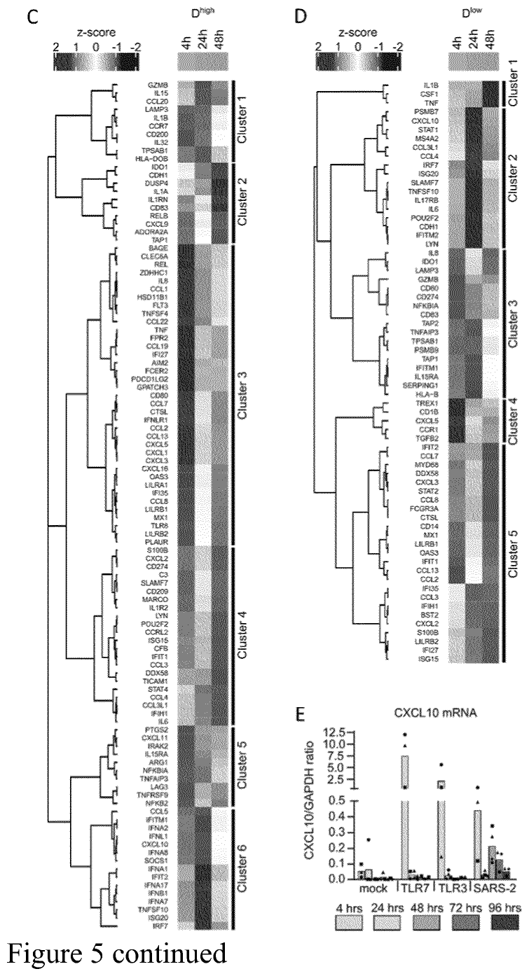

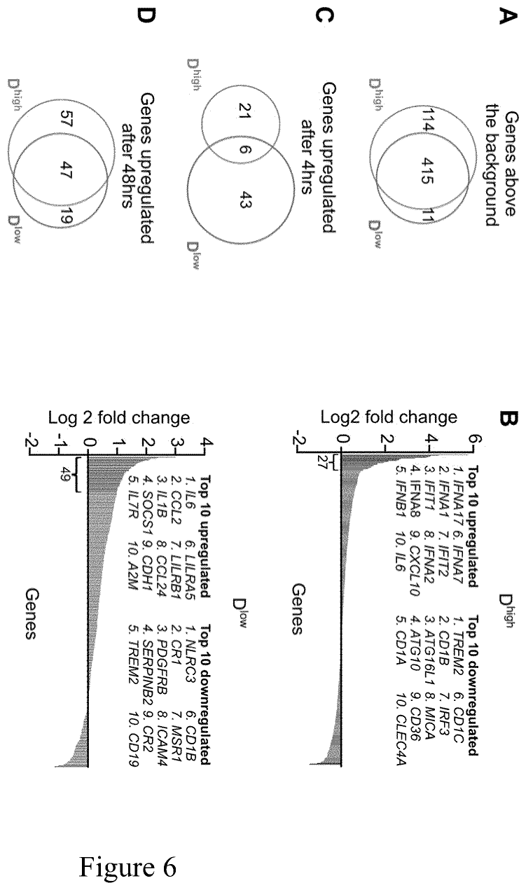

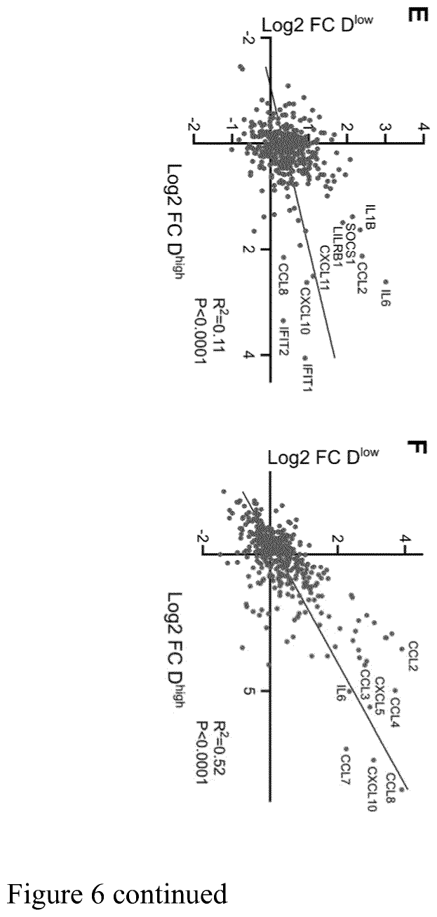

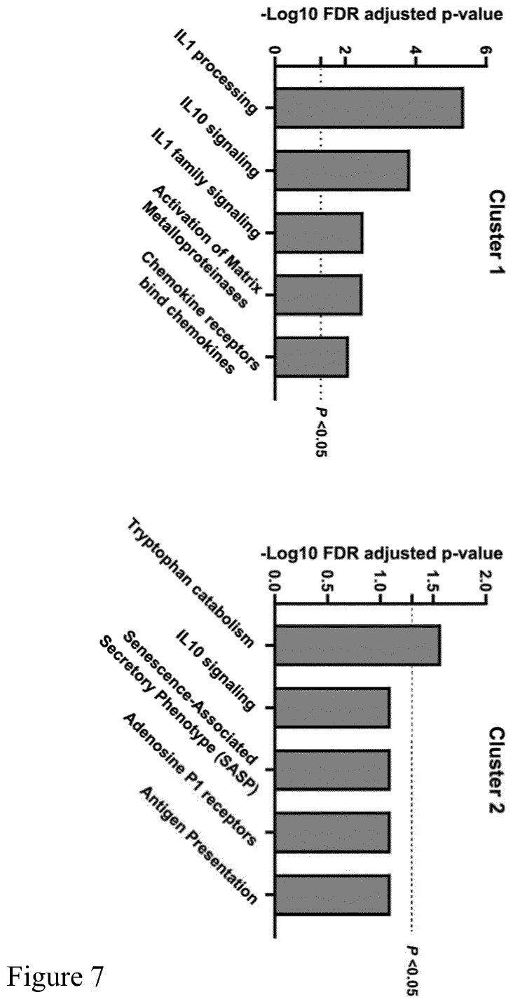

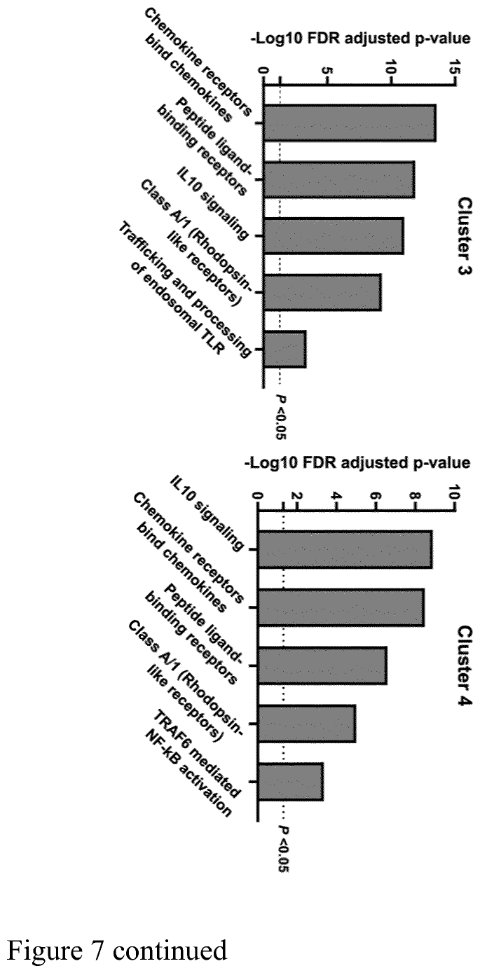

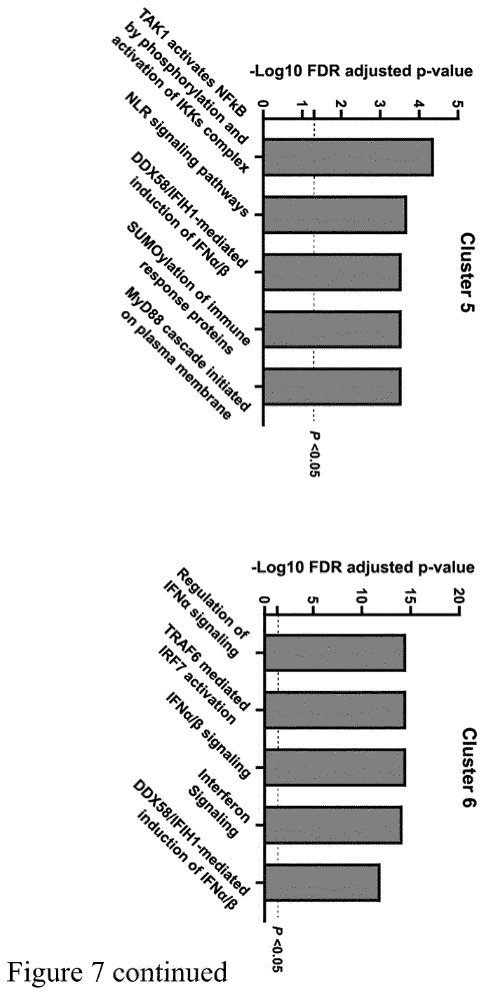

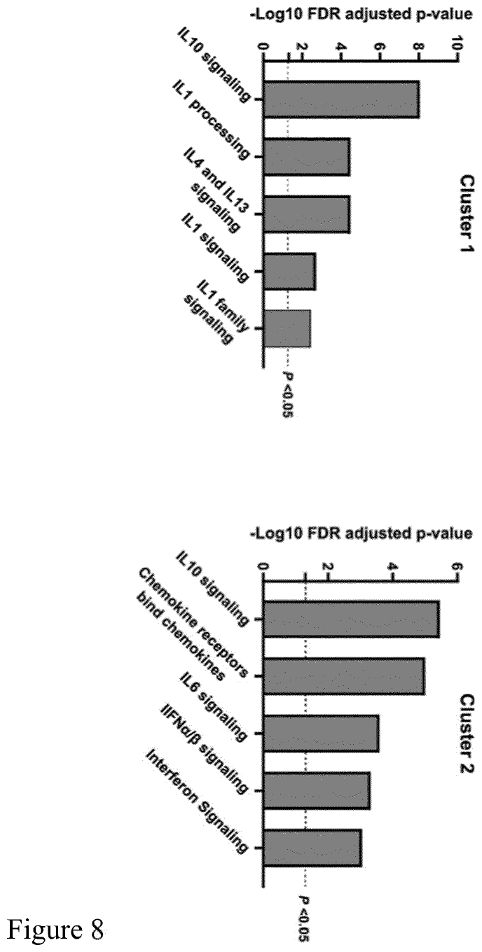

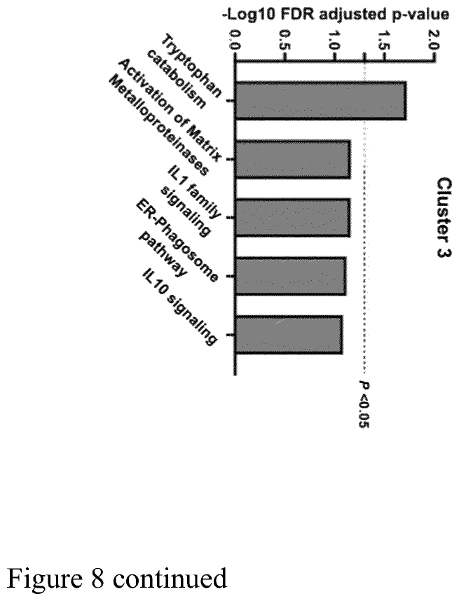

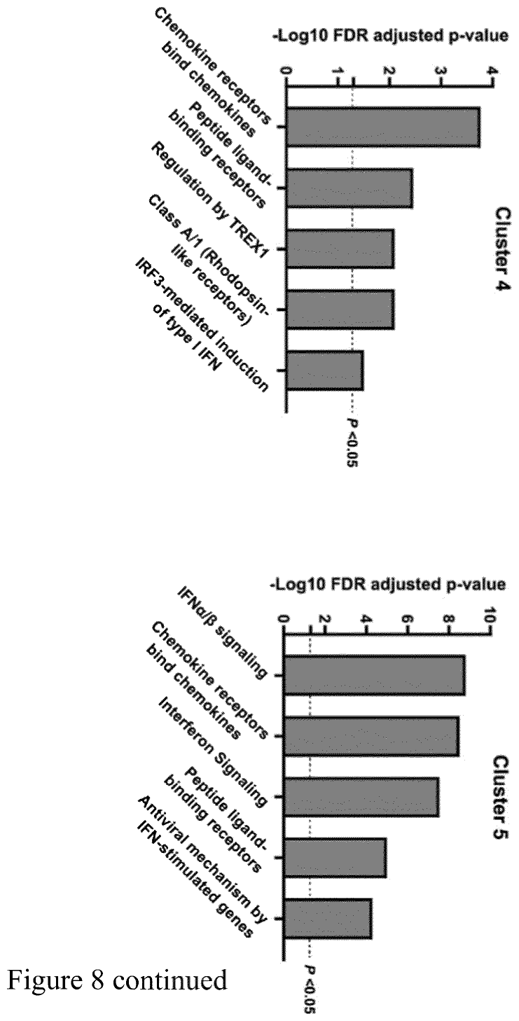

To broadly investigate the nature and timing of SARS-CoV-2-induced changes in pDC gene expression, we next profiled 789 selected genes covering major immunological pathways using the NanoString nCounter technology (22). We profiled the selected genes 4, 24 and 48 hrs after SARS-CoV-2 infection in two individual donors found to be high (D^{high}) and low (D^{low}) responders in terms of type I IFNα production (see FIG. 2A, where triangles denote D^{high} and squares denote D^{low}). There was a large overlap between the two donors in gene expression detected above background levels (FIG. 6A) and while multiple genes were induced as early as 4 hrs post SARS-CoV-2 exposure, the immunological response seemed stronger after 48 hrs (FIG. 5A-B, FIG. 6B-D). Interestingly, IL-6, CXCL10, CCL2 and CCL8 were among the most upregulated genes in both donors after both 4 and 48 hrs (FIG. 6E-F). When comparing the expression of the most strongly induced genes (fold change>2, relative to mock treated cells) after 48 hours of infection (104 genes in D^{high} and 66 genes in D^{low}) with the earlier time points, it became apparent that distinct gene sets behaved differently (FIG. 5C-D). For instance, some genes peaked at 4 hrs or 24 hrs, some clearly peaked at 48 hrs (cluster 4 FIG. 5C and cluster 5 FIG. 5D), while others had a clear biphasic expression format (induced early, disappearing after 24 hrs and then re-induced after 48 hrs; cluster 6 FIG. 5C and cluster 2 FIG. 5D). These gene clusters included pathways involved in the pDCs' anti-viral response, and pathways representing more general innate immune activation (FIG. 7 FIG. 8). To confirm the intriguing biphasic gene induction, CXCL10 gene expression was selected for further analysis in multiple pDC donors. RT-qPCR analysis revealed that the wave pattern of gene induction was specific for SARS-CoV-2 sensing and did not follow a TLR7 or TLR3 induction pattern (FIG. 5E), confirming our previous observations. Overall, this demonstrates that SARS-CoV-2 activates different steps at different time points in the pDCs' viral sensory pathways, indicating a multifaceted sensory mechanism, where antiviral type I IFNα is found in the early phase, succeeded by excessive inflammatory cytokines at later times. This reflects in part the pathogenesis of COVID-19 (9).

### MyD88 is Required for Interferon Responses to SARS-CoV-2

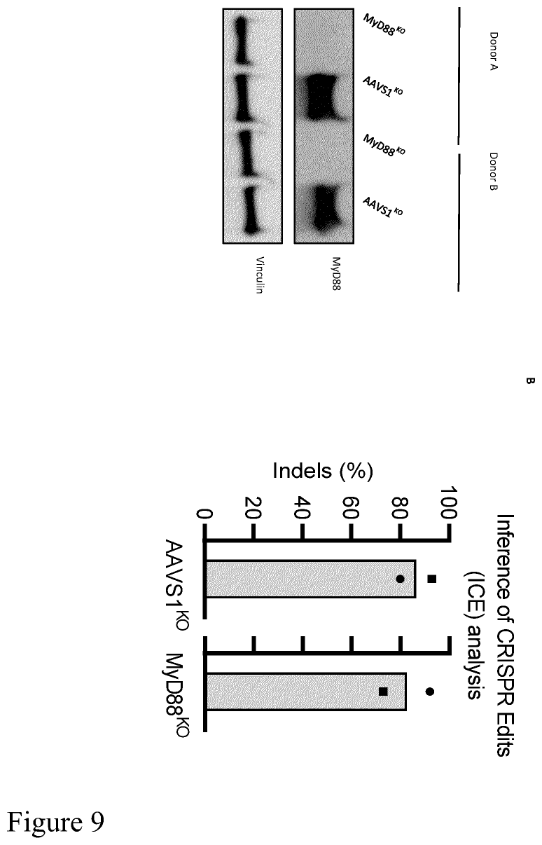

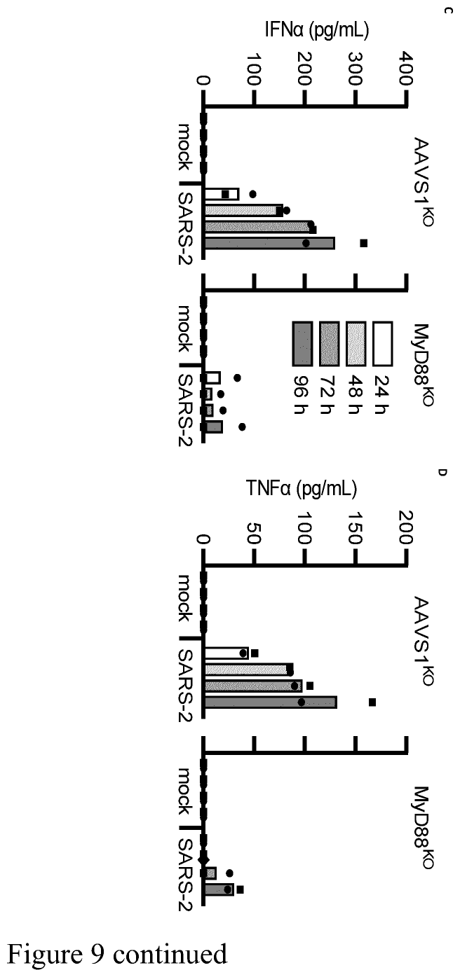

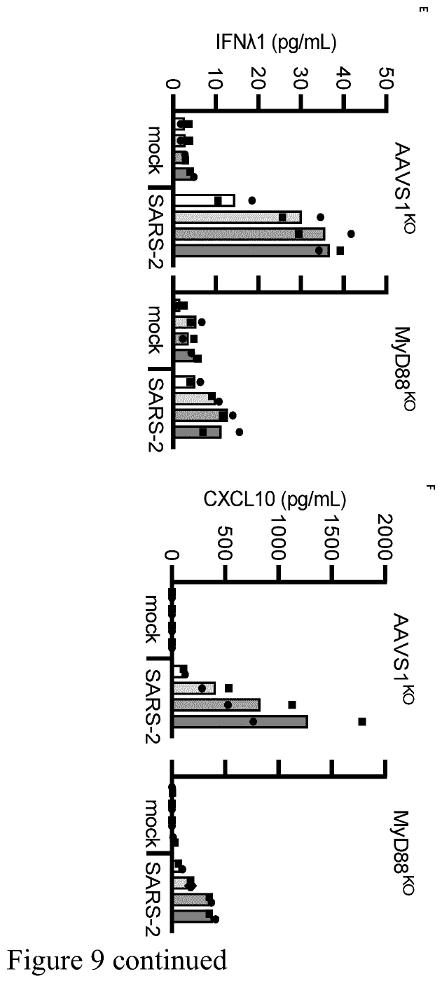

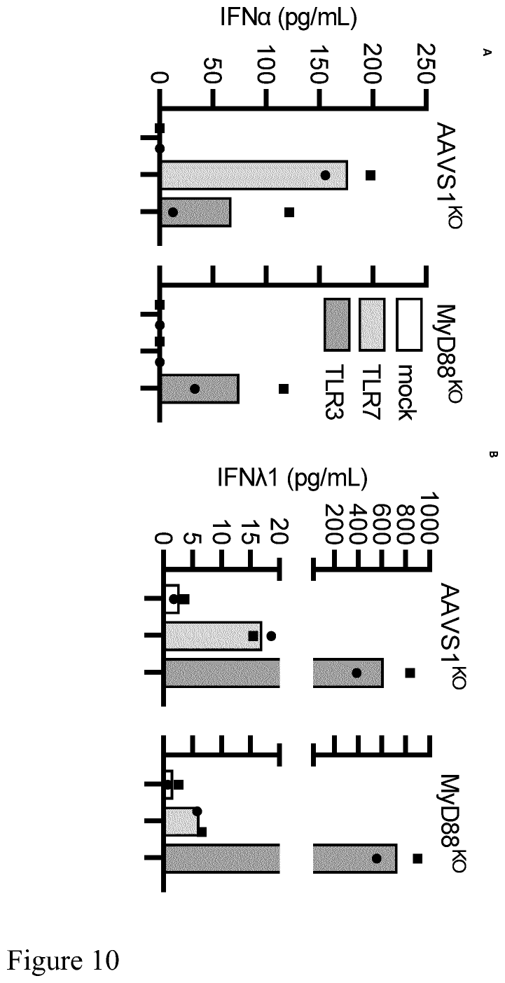

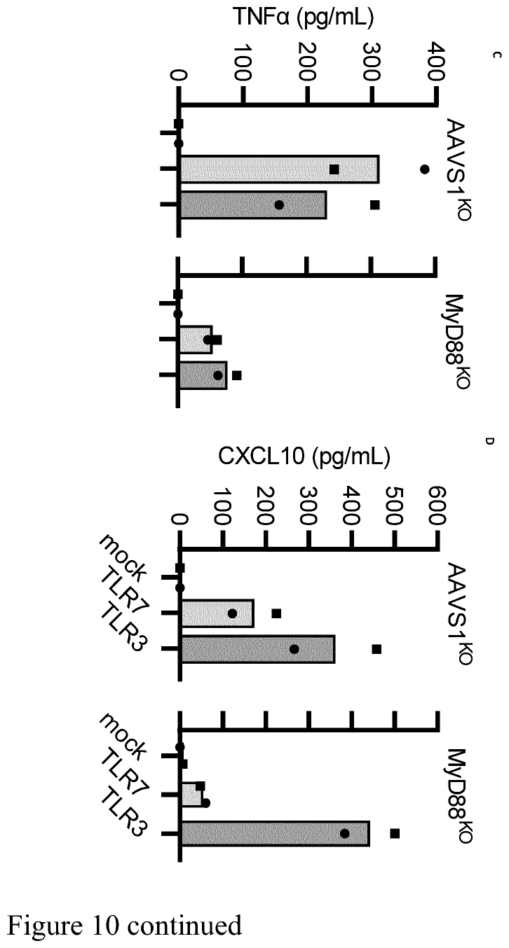

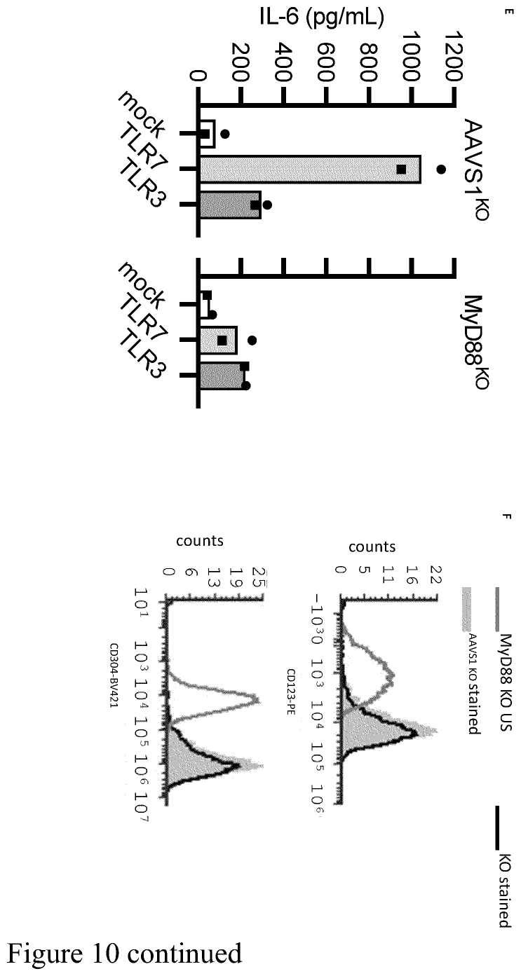

SARS-CoV-2—a single stranded RNA virus—may potentially be sensed by the endosomal TLR-MyD88 (Myeloid differentiation primary response 88) pathway (3, 4, 15). To evaluate this in detail, we first generated MyD88 knockout (MyD88^{KO}) pDCs using CRISPR/Cas9. As a control, we included cells targeted with CRISPR at the inert (safe-harbor) genomic locus AAVS1 (AAVS1^{KO}). MyD88 knockout was confirmed by protein expression (FIG. 9A), inference of CRISPR edits (ICE) analysis (FIG. 9B), as well as lack of type I IFNα and cytokine induction in response to TLR7 agonist stimulation (FIG. 10A-E). Of note, knockout of MyD88 did not affect the pDC phenotype (FIG. 10F). When exposed to SARS-CoV-2, we observed that MyD88^{KO} pDCs were severely impaired in the induction of CXCL10 and type I IFNα (FIG. 9C-D).

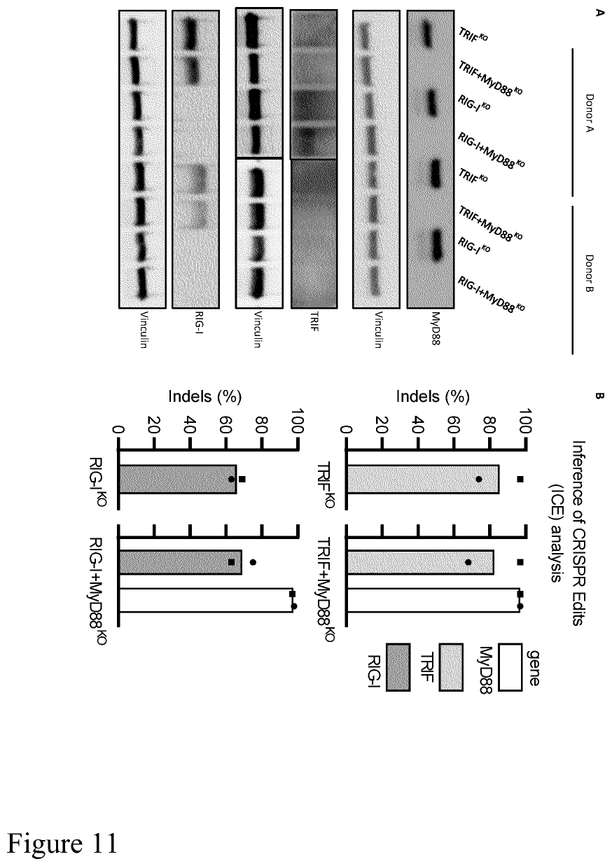

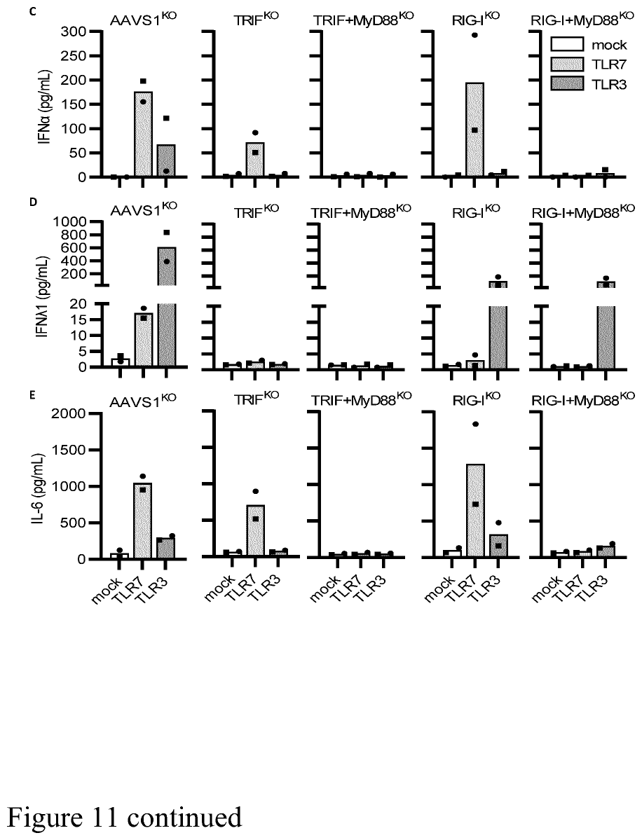

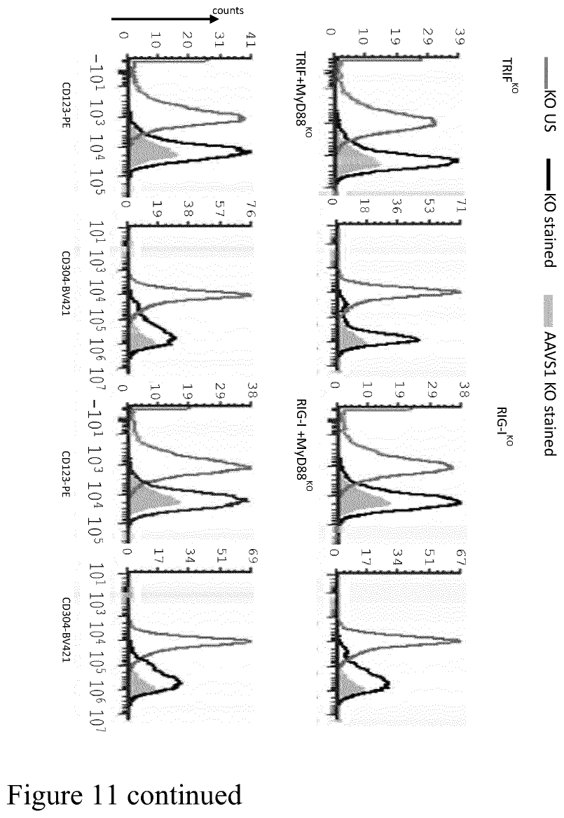

Different RNA sensing mechanisms can be active in pDCs and potentially sense SARS-CoV-2; the endosomal TLR3-TRIF (TIR-domain-containing adaptor-inducing IFNβ) cascade and the intracellular RIG-I-MAVS (retinoic acid-inducible gene I/pathway—mitochondrial antiviral signaling protein) (23-25). Though TLR3 predominantly binds short double stranded (ds)RNA, it can also bind regions found in secondary RNA structures such as loops and bulges (26). As pDCs have been reported to express TLR3, albeit at lower levels than classical myeloid DCs (24), we next generated and validated pDCs with a TRIF^{KO} and a double TRIF+MyD88^{KO} (FIG. 11). Disrupting TRIF signaling impaired agonist-induced IFNλ1 production (FIG. 11C) but did not affect type I IFNα production in response to SARS-CoV-2 exposure (FIG. 9E). Next, we tested the RIG-I pathway, which is both expressed and further upregulated in pDCs upon TLR stimulation and type I IFN signaling (23, 25). Disrupting RIG-I signaling showed a similar response pattern as observed for the TRIF^{KO} pDCs exposed to SARS-CoV-2, indicating this pathway is not involved in the sensing of SARS-CoV-2 and subsequent type I IFNα production by pDCs (FIG. 9F, FIG. 11). Altogether, our results indicate that pDCs primarily sense SARS-CoV-2 and induce antiviral cytokine production via a MyD88 controlled pathway.

TLR7 and TLR2 Sense SARS-CoV-2 with Divergent Inflammatory Responses.

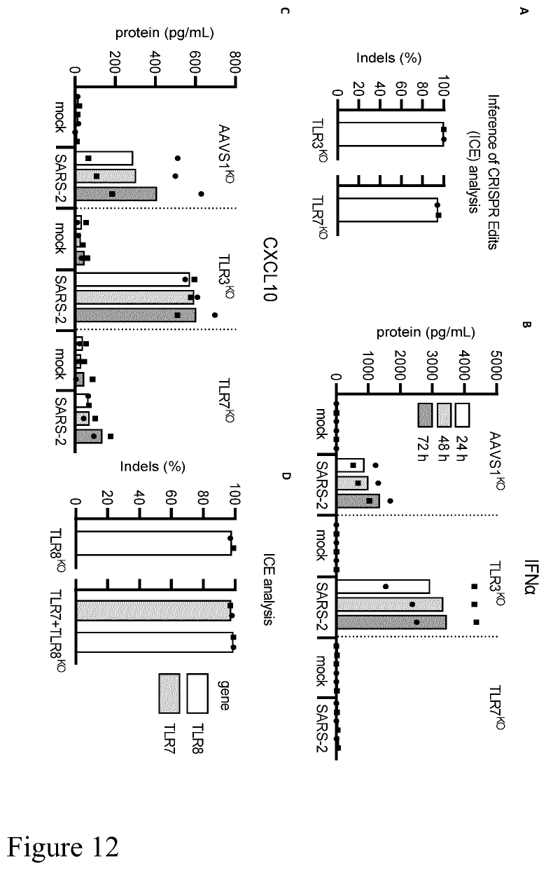

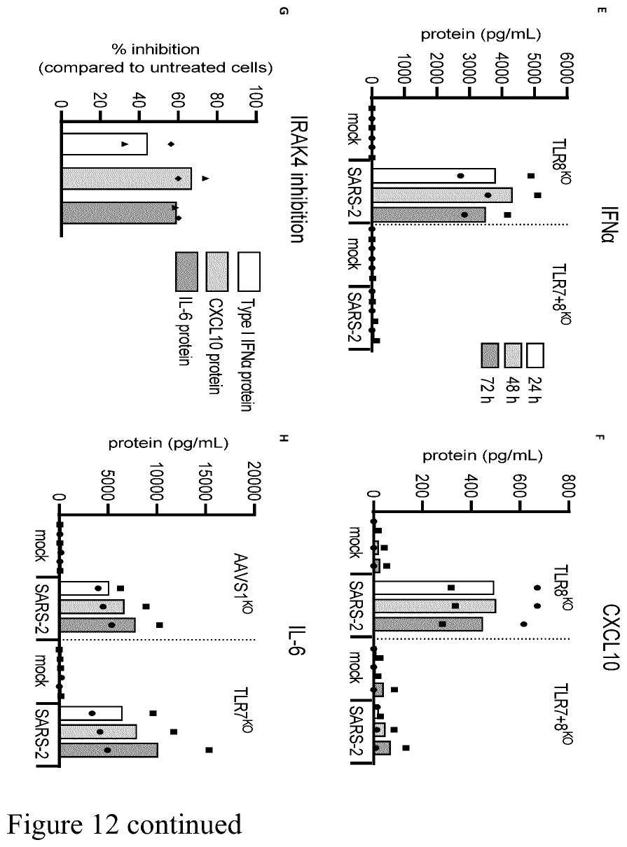

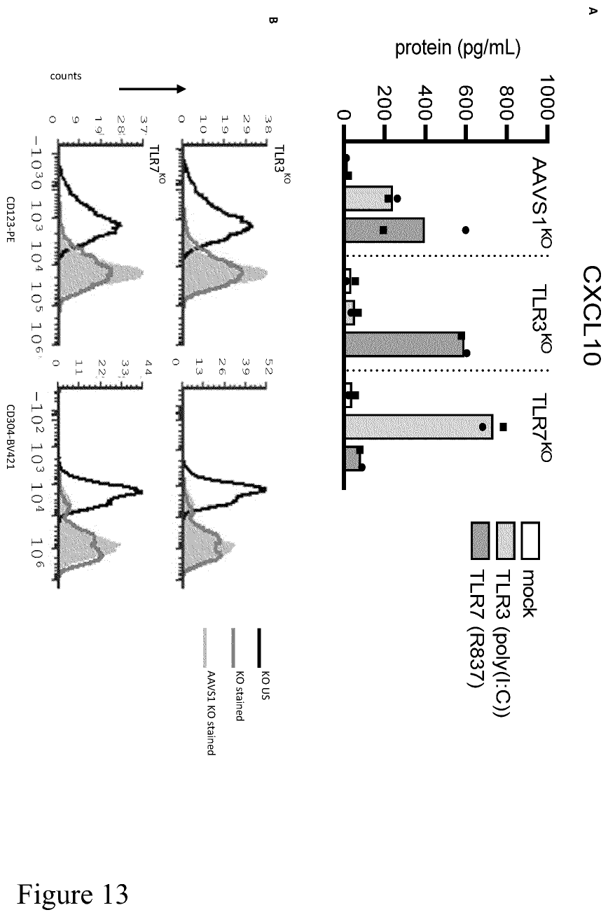

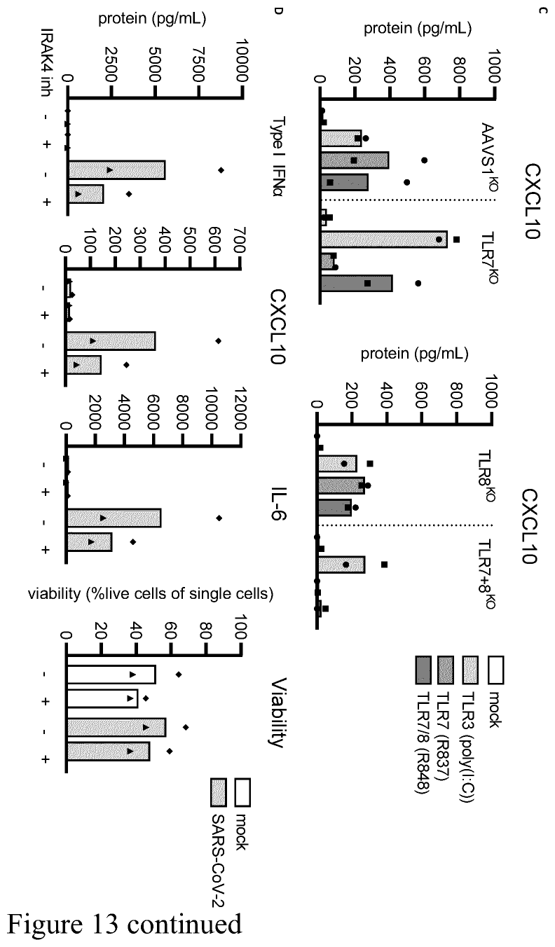

To narrow down the TLR responsible for sensing SARS-CoV-2 and controlling the induction of cytokines, we next generated pDCs with TLR3^{KO} or TLR7^{KO}. Disrupting these two pattern recognition receptors (FIG. 12A, FIG. 13A-B) clearly demonstrated that TLR3 was not involved in the production of type I IFNα and CXCL10 post sensing of SARS-CoV-2 (FIG. 12B-C). However, TLR7 knockout completely abolished type I IFNα and severely impaired CXCL10 production in response to SARS-CoV-2 exposure (FIG. 12B-C). Disruption of TLR8, another intracellular viral RNA sensor, with and without TLR7^{KO} confirmed that type I IFNα production in response to SARS-CoV-2 was solely driven by TLR7 (FIG. 12D-F, FIG. 13C). Inhibition of the Interleukin 1 Receptor Associated Kinase 4 (IRAK4), previously shown to be important for SARS-CoV-2-induced cytokine induction in pDCs (21), reduced type I IFNα, CXCL10 and IL-6 protein production upon SARS-CoV-2 sensing, without major effects on cell viability (FIG. 12G, FIG. 13D). Remarkably, we observed that SARS-CoV-2-induced IL-6 production was unaffected by the disruption of the TLR7 sensing pathway (FIG. 12H).

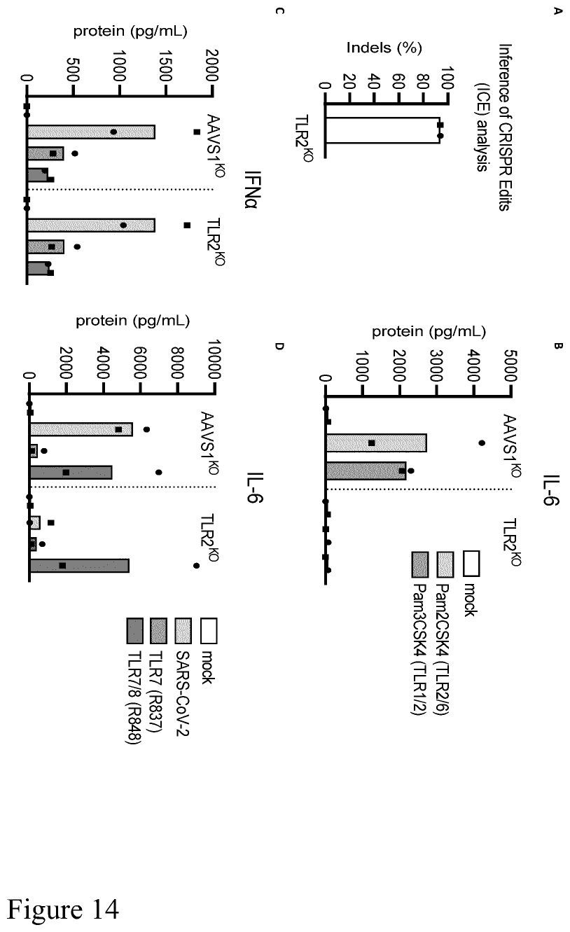

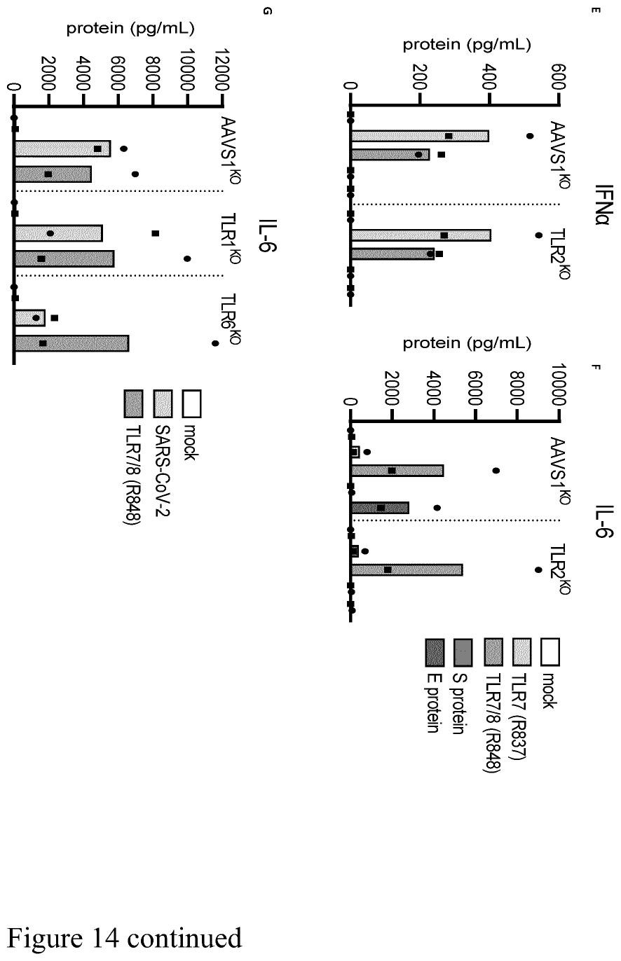

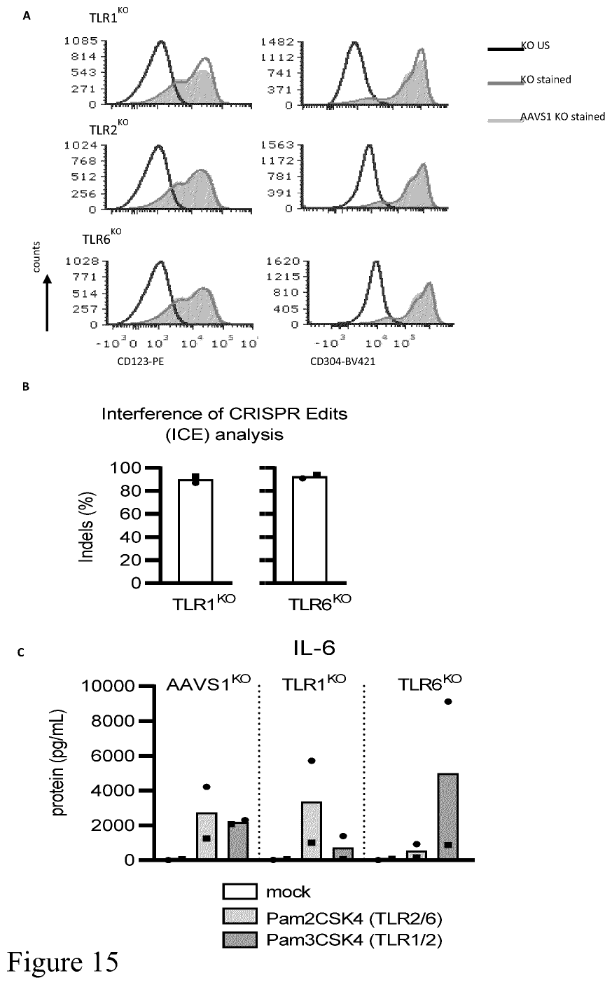

Multiple studies have shown that elevated levels of IL-6 in COVID-19 patients are associated with disease severity (7, 8, 27, 28) and thus we next focused on determining what sensing mechanism was responsible for the IL-6 production by pDCs. As murine bone marrow-derived macrophages and human PBMCs can utilize TLR2 to detect SARS-CoV-2 envelope protein (29) we hypothesized that this TLR could be engaged by human pDCs to sense SARS-CoV-2 and produce IL-6. First, we generated TLR2^{KO} HSPC-pDCs (FIG. 14A-B) where disruption of TLR2 did not affect SARS-CoV-2-mediated type I IFNα production (FIG. 14C), but did severely impair IL-6 production (FIG. 14D). Using recombinant glycoproteins of SARS-CoV-2 we found that the TLR2 sensing was driven by envelope protein but not the viral spike protein (FIG. 7E-F). Importantly, TLR2 is known to form heterodimers with either TLR1 or TLR6 (30) suggesting that these receptors could be involved in SARS-CoV-2 envelope protein sensing. Here, TLR6^{KO} pDCs but not TLR1^{KO} pDCs displayed a disrupted IL-6 production in response to SARS-CoV-2 exposure (FIG. 14G, FIG. 15A-C), indicating that pDCs produce IL-6 in response to TLR2 and TRL2/6-mediated sensing of SARS-CoV-2.

SARS-CoV-2 Uses Neuropilin-1 to Evade the pDCs' Anti-Viral Response.

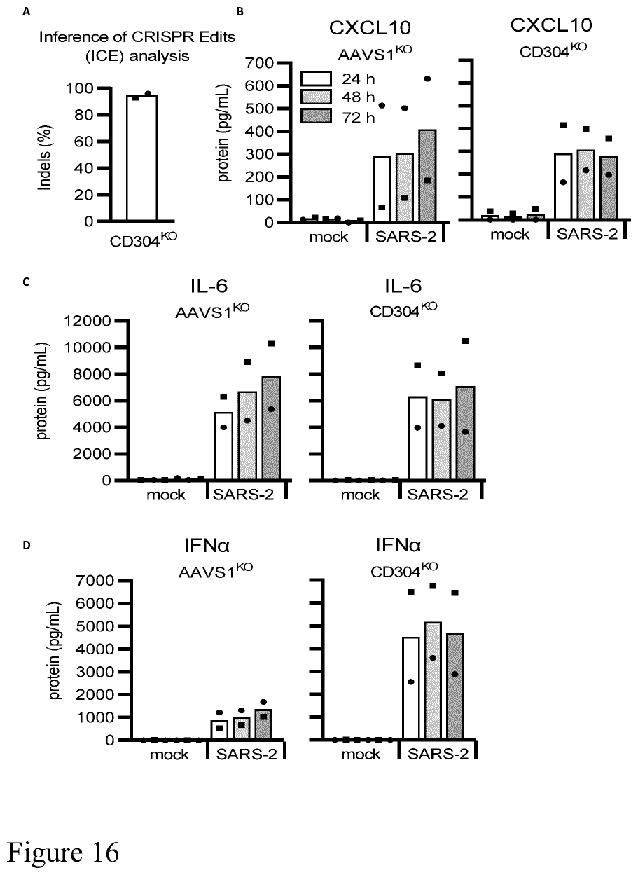

A few papers have recently shown that SARS-CoV-2 can bind to neuropilin-1/CD304/BDCA-4 as alternative to ACE2 for viral entry (31, 32). Interestingly, neuropilin-1 is one of the phenotypic markers for pDCs and has a functional role in pDC biology by reducing IRF7-mediated type I IFNα production (33, 34). To investigate if SARS-CoV-2 uses neuropilin-1 to mitigate the type I IFNα response by pDCs, we finally generated CD304^{KO} HSPC-pDCs (FIG. 16A). Notably, both CXCL10 and IL-6 production upon SARS-CoV-2 sensing was not affected in CD304^{KO} pDCs (FIG. 16B-C), however, type I IFNα responses increased drastically 4-6 fold (FIG. 16C). This clearly indicates that CD304-receptor activation impairs the type I IFNα production by pDCs upon SARS-CoV-2 sensing, which suggest that SARS-CoV-2 reduces the pDCs' type I IFNα production by using neuropilin-1 as an immune evasion strategy.

### CONCLUSION

CRISPR/Cas9-editing of human stem cell-derived pDCs demonstrated that pDC sense SARS-CoV-2 and produce different pro-inflammatory cytokines in response. The viral E glycoprotein is recognized by the extracellular TLR2/6 heterodimer, leading to an IRAK4 dependent production of the pro-inflammatory IL-6 cytokine. The intracellular TLR7-MyD88-IRAK4 pathway facilitates the production of CXCL10 and antiviral type I IFNα, of which the latter can protect lung epithelial cells from de novo SARS-CoV-2 infection. Removing the CD304-induced inhibition on IRF7 translocation, revealed that type I IFNα but not CXCL10 production is dependent on this interferon response factor. Overall, we show that pDCs can sense SARS-CoV-2 and induce a strong antiviral response that is sufficient to protect lung epithelial cells from de novo SARS-CoV-2 infection, even though SARS-CoV-2 utilizes an intrinsic immune evasion strategy that mitigates antiviral IFN production.

### DISCUSSION

COVID-19 severity is associated with the excessive production of inflammatory cytokines, also described as a ‘cytokine storm’, yet which cells produce these cytokines succeeding SARS-CoV-2 infection is not fully understood. Our findings show that pDCs, an immune cell type important for the host defense against many viruses, efficiently detect SARS-CoV-2 by a multi-faceted sensing mechanism and in response produce inflammatory and antiviral cytokines, including type I IFNα and IL-6.

Since SARS-CoV-2 emerged, multiple studies have suggested that different cell types as well as diverging sensing pathways to be responsible for the control of the viral infection and the increased levels of inflammatory cytokines observed in patients. One of the challenges by exploring the antiviral response of pDCs is the limited number of cells to collect from blood and the notorious difficulties to genetically manipulate these cells. This can partly be overcome by collecting pDCs from patients with genetic disorders (21) or by studying mice. However, some TLR pathways have been reported to either being nonfunctional or controversial in mice models (35, 36). In the present study, using a stem cell-based human pDC model in combination with CRISPR KO of multiple TLRs and signaling factors, we demonstrated that TLR7 is critical for the inflammatory signal induced by SARS-CoV-2 infection. Unexpectedly, reduction of the inflammatory cytokine IL-6 was solely dependent on the TLR2 pathway whereas TLR7-MyD88 was responsible for the remaining inflammatory cytokines. More studies in pDCs will be needed to understand how these signaling pathways are functioning molecularly.

Highly pathogenic coronaviruses, similar to other viruses, have multiple strategies to interfere with the host's immune response and efficient immune evasion is associated with pathogenicity (19). Therefore, a detailed understanding of SARS-CoV-2's immune evasion strategies is critical for the development of antiviral therapeutics. Our data indicate that SARS-CoV-2 utilizes neuropilin-1 not only as alternative receptor to ACE2 for viral entry, but also to mitigate the production of type I IFNα by pDCs, thereby reducing the host's innate antiviral immune response. This may call for future studies to explore whether neuropilin-1 blocking antibodies will be a clinically applicable treatment to increase the antiviral response supported by pDCs and thereby dampen the viral spread.

As pDCs support both the rapid type I IFNα secretion and IL-6 production, this suggests that these cells may have a double-edged function during COVID-19 pathogenesis. Without active pDCs in the lungs, antiviral protection may not be mounted, whereas sustained pDC activation could exacerbate lung inflammation via IL-6 production. Blocking IL-6 responses may not necessarily be successful clinically but therapy with antagonists that specifically impair TLR2, and not TLR7, or therapeutics targeting the viral E glycoprotein could potentially be a scenario to direct immune cells, such as pDCs, to mount a stronger type I and III IFN response that could mitigate disease pathogenesis.

In conclusion, our study provides evidence that circulating pDCs could be a potential therapeutic target to maintain desired antiviral IFN levels allowing for the mitigation of COVID-19 severity.

### DETAILED DESCRIPTION OF FIGURES

FIG. 1—shows evidence that plasmacytoid DCs can sense SARS-CoV-2 and induce an inflammatory response. To evaluate whether pDCs sense SARS-CoV-2 and induce an inflammatory response, HSPC-pDCs were either mock treated or exposed to the SARS-CoV-2 FR2020 early Wuhan-like strain or the SARS-CoV-2 alpha variant (MOI 0.1). Supernatants were collected at indicated time points and the production of type I IFNα (A) and CXCL10 (B) was quantified.

To assess a broader range of SARS-CoV-2-induced cytokines by pDCs, the FR2020 strain was used in subsequent experiments. HSPC-pDC were either mock treated (mock), exposed to SARS-CoV-2 (MOI 1), TLR7 (2.5 μg/mL R837) or TLR3 agonist (800 ng/mL poly(I:C)). Supernatants were collected after 24 h and analyzed for type I IFNα (C), IFNβ (D), type II IFNγ (E), type III IFNλ1 (F), IL-6 (G), IL-8 (H), CXCL10 (I) and TNFα (J) expression by ELISA. To evaluate the cytokine response to viral titers and exposure duration, HSPC-pDCs were exposed to increasing viral inoculums (MOI 0.01, 0.1 and 1) and IFNα2a mRNA expression was quantified at 24 h (K) and IFNα protein secretion at 24 h, 48 h, 72 h and 96 h (FIG. 2A). Graph depicting simple linear regression of IFNα protein with time of exposure (M).

Bars and lines represent mean values and symbols represent individual HSPC-pDC donors (n=2-4). Equal symbols represent equal donors (A-B and C-J). Statistical significance was determined using the ratio paired student T test and simple linear regression. *<p0.05, **<p0.01***<p0.001.

FIG. 2—shows evidence that induction of inflammatory cytokines enhances with increasing viral titers and duration of exposure. HSPC-pDCs were either mock treated (mock) or inoculated with increasing SARS-CoV-2 titers (MOI 0.01, 0.1 or 1). Supernatant was collected after 24 h, 48 h, 72 h and 96 h for the quantification of IFNα (A), type III IFNλ1 (B), IL-6 (C), IL-8 (D), CXCL10 (E) and TNFα (F) proteins. Bars represent mean values, symbols represent individual HSPC-pDC donors (n=2-4). Equal symbols represent equal donors.

FIG. 3—shows evidence that plasmacytoid DCs are not a reservoir for SARS-CoV-2 replication. To assess whether SARS-CoV-2 replicates in pDCs, HSPC-pDCs were mock treated (mock) or exposed to increasing SARS-CoV-2 titers (MOI 0.01, 0.1 or 1). Supernatants (A) and cells (B) were collected at different time intervals, as indicated, and viral outgrowth in supernatant (A) and intracellular viral amplification (B) was analyzed using the limiting dilution assay and qPCR, respectively. Virus in cell culture supernatant was normalized to viral titers at the 24 h time point. To confirm that the utilized assays detected SARS-CoV-2 replication, air liquid interface (ALI) epithelial cell cultures were used as a control for productive infection of SARS-CoV-2 (MOI 0.5) and viral outgrowth (C) as well as intracellular SARS-CoV-2 amplification (D). ACE2 expression was measured on HSCP-pDC and VeroE6 TMPRSS2 by flow cytometry (E). Quantification of ACE2 (F) and TMPRSS2 (G) mRNA in HSPC-pDC and epithelial cells (not detectable was set to 0). Bars represent mean values, symbols represent individual HSPC-pDC donors (n=3-4).

FIG. 4—shows evidence that cytokines secreted by plasmacytoid DCs after sensing of SARS-CoV-2, protect lung epithelial cells from de novo SARS-CoV-2 infection. To assess whether pDCs could mount protection against SARS-CoV-2, conditioned medium from SARS-CoV-2-exposed pDC cultures (d3 post inoculation with MOI 1) was added to A549 hACE2 lung epithelial cells (A) or Calu-3 (B) cultures followed by SARS-CoV-2 inoculation. The cell cultures were conditioned with normal medium (-), pDC supernatant (pDC mock) or SARS-CoV-2-inoculated pDC supernatant (pDC SARS-2), prior to infection with SARS-CoV-2 (MOI 0.1). Supernatants were collected and viral outgrowth was determined 48 h post infection. To investigate a potential dose-response, SARS-CoV-2-inoculated pDC supernatant was 3-fold serially diluted prior to addition to Calu-3 cells (B). To determine the involvement of type I IFNs, Calu-3 cells and SARS-CoV-2-inoculated pDC supernatants were pre-treated with antibodies blocking the type I IFN receptor and antibodies neutralizing type I IFNα (IFN-I block) or isotype control antibodies (isot ctrl), prior to the addition of conditioned medium to the cells and infection (C). Bars and lines represent mean values and symbols represent individual HSPC-pDC donors (n=3) Equal symbols represent equal donors. Statistical significance was determined using the ratio paired student T test. *<p0.05, ***<p0.001.

FIG. 5—shows evidence of the nature and timing of SARS-CoV-2-induced gene expression changes in pDCs. Waterfall plots illustrating gene expression changes in pDCs 48 h post SARS-CoV-2 exposure, relative to mock treated cells, from two donors; D^{high} (A) and D^{low} (B), indicating genes with >2 fold change in red (upregulated) and blue (downregulated). Heat maps and unsupervised hierarchical cluster analyses of the >2 fold upregulated genes in D^{high} (C) and D^{low} (D). To validate the wave pattern of gene expression, CXCL10 mRNA was quantified using RT-qPCR in multiple pDC donors at different time points post TRL7 agonist R837, TLR3 agonist poly(I:C) and SARS-CoV-2 (1 MOI) exposure at indicated time points. Bars represent mean values and equal symbols represent equal donors (n=2−4).

FIG. 6—shows evidence of SARS-CoV-2-induced gene expression changes in pDCs. Venn diagram illustrating the large overlap in gene expression detected above background levels in D^{high} and D^{low}, measured using a NanoString nCounter (A). Waterfall plots illustrating gene expression changes in pDCs 4 h after SARS-CoV-2 exposure relative to mock treated cells from two donors; D^{high} (B, top) and D^{low} (B, bottom). Venn diagrams illustrating the number of genes that were >2 fold upregulated after 4 h (C) and 48 h (D) in D^{high} and D^{low} post SARS-CoV-2 exposure. Scatter plots of log 2 fold changes (FC) in gene expression in D^{high} and D^{low} after 4 h (E) and 48 h (F) post SARS-CoV-2 inoculation with corresponding linear regression statistics and R-squared values. Note that the genes, which were not detected above background levels in any of the donors, could not be displayed due to division by zero.

FIG. 7—shows a Reactome Pathway Analysis for donor D^{high}. Histograms illustrating the five most significant pathways and the associated False Discovery Rate (FDR) corrected P-values for the gene clusters derived from the unsupervised hierarchical clustering analysis displayed in FIG. 4C for D^{high}. Reactome uses the Binomial Test to assess the probability that the overlap between the query and the pathways has occurred by chance and the FDR is calculated using the Benjamini-Hochberg approach. Dotted lines indicate statistical significance. Antigen representation: Folding, assembly and peptide loading of class I MHC.

FIG. 8—shows a Reactome Pathway Analysis for donor D^{low}. Histograms illustrating the five most significant pathways and the associated False Discovery Rate (FDR) corrected P-values for the gene clusters derived from the unsupervised hierarchical clustering analysis displayed in FIG. 4D for D^{low}. Reactome uses the Binomial Test to assess the probability that the overlap between the query and the pathways has occurred by chance and the FDR is calculated using the Benjamini-Hochberg approach. Dotted lines indicate statistical significance.

FIG. 9—shows evidence that SARS-CoV-2 sensing and inflammatory cytokine induction by pDCs is mediated predominantly via MyD88. Using CRISPR/Cas9, MyD88 knock-out (KO) and AAVS1^{KO} (control) HSPC-pDCs were generated. MyD88 protein levels in KO and control pDCs were analyzed by western blotting (A) and cellular DNA was sequenced to perform an Inference of CRISPR Edits (ICE) analysis (B). MyD88^{KO} and control pDCs were either mock treated (mock) or exposed to SARS-CoV-2 (SARS-2, MOI 1), supernatants were collected at indicated time points and analyzed for CXCL10 (C) and type I IFNα (D). Type I IFNα production was then determined in cell culture supernatant from SARS-CoV-2 exposed TRIF^{KO} or TRIG+MyD88^{KO} (E) and RIG-I^{KO} or RIG-I+MyD88^{KO} (F) pDCs. Bars represent mean values and equal symbols represent equal donors (n=2).

FIG. 10—shows validation of MyD88^{KO} HSPC-pDCs. HSPC-pDC-MyD88^{KO} were functionally evaluated by stimulating the cells with TLR7 (2,5 ug/mL R837, central bars) or TLR3 (800 ng/mL poly(I:C), right hand bars) agonist and collecting supernatant 24 h after stimulation to quantify IFNα (A), IFNλ1 (B), TNFα (C), CXCL10 (D) and IL-6 (E) protein concentrations. HSPC-pDC-MyD88^{KO} were phenotypically evaluated for expression of the pDC markers CD123 and CD304, and compared to unstained (US) HSPC-pDC-MyD88^{KO} and stained HSPC-pDC-AAVS1^{KO} (F).

FIG. 11—shows validation of TRIF^{KO} and RIG-I^{KO} HSPC-pDCs. Knock out efficiency was evaluated at the protein level by western blot analysis for MyD88 (A, top panel), TRIF (A, middle panel) and RIG-I (bottom panel), and by genomic sequencing and ICE analysis to assess the frequency of insertion and deletions (indels) at the targeted sites (B). The TRIF antibody functionality was confirmed with Thp-1 cell lysates (data not shown), yet we were unable to detect TRIF protein in donor B. Additionally, HSPC-pDC^{KO} cells were evaluated functionally by stimulating the cells with TLR7 (2,5 ug/mL R837, central columns) or TLR3 (800 ng/mL poly(I:C), right columns) agonist and collecting supernatant after 24 h to quantify IFNα (C), IFNλ1 (D) and IL-6 (E) protein concentrations. HSPC-pDC^{KO} were phenotypically evaluated for expression of the pDC markers CD123 and CD304, and compared to unstained (US) KO cells and stained control AAVS1^{KO} HSPC-pDC (F). Bars represent mean values and equal symbols represent equal donors (n=2, donor A is represented by the circle and Donor B by the square symbol).

FIG. 12—shows evidence that SARS-CoV-2 sensing and subsequent IFNα production by pDCs is solely mediated by TLR7. SARS-CoV-2 sensing and subsequent type I IFNα production by pDCs is solely mediated by TLR7. Using CRISPR/Cas9, TLR3 and TLR7 knock-out (KO) and AAVS1^{KO} (control) HSPC-pDCs were generated and cellular DNA was sequenced for ICE analysis (A). AAVS1^{KO}, TLR3^{KO} and TLR7^{KO} pDCs were either mock treated (mock) or exposed to SARS-CoV-2 (SARS-2, MOI 1), supernatants were collected at indicated time points and analyzed for type I IFNα (B) and CXCL10 (C) proteins. To determine if the intracellular RNA sensor TLR8 can detect SARS-CoV-2 and induce cytokine production, TLR8^{KO} and TLR7+TLR8^{KO} HSPC-pDCs were generated and inference of CRISPR Edits was analyzed by sequencing (D). TLR8KO and TLR7+TLR8KO pDC were either mock treated or exposed to SARS-CoV-2 (MOI 1) and cell culture supernatants were analyzed for type I IFNα (E) and CXCL10 (F) protein production. Wild type HSPC-pDC were exposed to SARS-CoV-2 (MOI 0.5) in the absence or presence of an IRAK4 inhibitor, 24 hrs after virus exposure the cell culture supernatants were analyzed for production of type I IFNα, CXCL10 and IL-6 proteins (G). IL-6 protein quantification in AAVS1^{KO} and TRL7^{KO} pDCs after SARS-CoV-2 exposure (MOI 1) at indicated time points (H). Bars represent mean values and equal symbols represent equal donors (n=2).

FIG. 13—shows validation of TLR3^{KO}, TLR7^{KO}, TLR8^{KO} and TRL7+TLR8^{KO} HSPC-pDCs. A: Functional evaluation the TLR3^{KO} and TLR7^{KO} pDCs. HSPC-pDCs were stimulated with TLR7 (2,5 ug/mL R848, right columns) or TLR3 (800 ng/mL poly(I:C), central columns) agonist, supernatant was collected after 24 h and analyzed for CXCL10 protein expression by ELISA. B: Phenotypic evaluation of HSPC-pDC^{KO} by flow cytometry. Histograms showing the expression of pDC markers CD123 and CD304, as compared to unstained KO pDCs and stained AAVS1^{KO} control pDCs. C: Functional evaluation the TLR7^{KO}, TLR8^{KO} and TLR7+TLR8^{KO} pDCs. HSPC-pDCs were stimulated with TLR7 (2,5 ug/mL R837, columns second from right), TLR3 (800 ng/mL poly(I:C),columns second from left) or TLR7/8 (2,5 ug/mL R848 right columns) agonist, supernatant was collected after 24 h and analyzed for CXCL10 protein expression by ELISA. D: Wild type HSPC-pDC were exposed to SARS-CoV-2 (MOI 0.5) in the absence or presence of an IRAK4 inhibitor (10 uM), 24 h after virus exposure the cell culture supernatants were analyzed for production of type I IFNα, CXCL10 and IL-6 proteins, and the cells were analyzed for viability with flow cytometry. Bars represent mean values and equal symbols represent equal donors (n=2).

FIG. 14—shows evidence that IL-6 production by pDCs is induced by TLR2/6-mediated sensing of the SARS-CoV-2 envelope protein. Using CRISPR/Cas9, TLR2^{KO} and AAVS1^{KO} (control) HSPC-pDCs were generated, cellular DNA was sequenced for ICE analysis (A) and cells were evaluated functionally by exposure to two different TLR2 agonists; Pam2CSK4 (5 ng/mL, central columns) and Pam3CSK4 (50 ng/mL, right columns) (B). Subsequently, AAVS1^{KO} and TLR2^{KO} pDCs were either mock treated (mock, left columns), exposed to SARS-CoV-2 (SARS-2, MOI 0.5,columns second from left), TLR7 (2.5 μg/mL R837,columns second from right) or TLR7/8 agonist (2.5 μg/mL R848, right columns) and supernatants were collected at indicated time points to quantify type I IFNα (C) and IL-6 (D) protein concentrations. To investigate if pDCs can sense the spike or envelope SARS-CoV-2 proteins, AAVS1^{KO} and TLR2^{KO} pDCs were exposed to TLR7 (2.5 μg/mL R837, columns second from left), TLR7/8 agonist (2.5 μg/mL R848, central columns), recombinant SARS-CoV-2 spike (S, 1 μg/mL, columns second from right) or envelope (E, 1 μg/mL, right columns) proteins and type I IFNα (E) and IL-6 (F) protein concentrations were quantified. TLR2 forms heterodimers with TLR1 and TLR6, to investigate which dimer is needed for SARS-CoV-2 sensing, AAVS1^{KO}, TLR1^{KO} and TLR6^{KO} pDCs were mock treated (left columns), exposed to SARS-CoV-2 (MOI 0.5, central columns) or TLR7/8 agonist (2.5 μg/mL R848, right columns) and IL-6 protein concentrations were quantified in cell culture supernatant after 24 hrs (G). Bars represent mean values and equal symbols represent equal donors (n=2).

FIG. 15—shows validation of TLR1^{KO}, TLR2^{KO} and TLR6^{KO} HSPC-pDCs. A: Phenotypic evaluation of HSPC-pDC^{KO} by flow cytometry. Histograms showing the expression of pDC markers CD123 and CD304, as compared to unstained KO pDCs and stained AAVS1^{KO} control pDCs. B: KO pDCs were evaluated by genomic sequencing followed by inference of CRISPR edits (ICE) analysis to assess the frequency of insertions and deletions (indels) at the target site. C: Functional evaluation the TLR1^{KO} and TLR6^{KO} pDCs. HSPC-pDCs were stimulated with TLR1/2 (50 ng/mL Pam3CSK4, right columns) or TLR2/6 (5 ng/mL Pam2CSK4, central columns) agonist, supernatant was collected after 24 h and analyzed for IL-6 protein expression by ELISA. Bars represent mean values and equal symbols represent equal donors (n=2).

FIG. 16—shows evidence that SARS-CoV-2 uses neuropilin-1 as immune evasion strategy by inhibiting type I IFNα production from pDCs. Inference of CRISPR Edits (ICE) analysis of CD304^{KO} HSPC-pDCs (A). AAVS1KO and CD304KO pDCs were either mock treated (mock) or exposed to SARS-CoV-2 (MOI 1), cell culture supernatants were collected at indicated times and analyzed for CXCL10 (B), IL-6 (C) and type I IFNα (D) protein concentrations. Bars represent mean values, equal symbols represent equal donors (n=2).

### Materials and Methods

### Cells

### HSPC-pDCs

HSPC-pDCs were generated as described previously (20). In brief, CD34⁺ HSPCs were purified from human umbilical cord blood (CB) acquired from healthy donors under informed consent from the Department of Gynecology and Obstetrics, Aarhus University Hospital, Aarhus. Mononucleated cells were recovered by standard Ficoll-Hypaque (GE Healthcare) density-gradient centrifugation and CD34⁺ cells were isolated using anti-CD34 immunomagnetic beads (positive selection) following the manufacturer's instructions (EasySep™ Human cord blood CD34⁺ positive selection kit II, STEMCELL Technologies Cat #17896). CD34⁺ HSPCs were either freshly used or cryo-preserved until future use. For HSPC to pDC differentiation, CD34⁺ HSPCs were cultured using serum free medium SFEM II (STEMCELL Technologies) supplemented with 20 U/mL penicillin and 20 μg/mL streptomycin (Penicillin-Streptomycin ThermoFisher Scientific), 100 ng/mL Flt3-L (Peprotech), 50 ng/mL TPO (Peprotech), 100 ng/mL SCF (Peprotech), 20 ng/mL IL-3 (Peprotech) and 1 μM SR1 (StemCell Technologies). Cells were cultured at 37° C., 95% humidity, and 5% C02, medium was refreshed every 3-4 days and cells were kept at a density of 0.5-5×10⁶ cells. After a 21-day differentiation period, pDCs were enriched using negative magnetic selection, according to the manufacturer's protocol (EasySep™ Human Plasmacytoid DC Enrichment kit, STEMCELL Technologies Cat #19062). Enriched HSPC-pDCs were then primed for 3 days in RF10 (RPMI-1640 medium (Merck) supplemented with 10% (v/v) heat-inactivated fetal calf serum (hiFCS, Sigma-Aldrich), 2 mM L-glutamine (ThermoFisher Scientific), 100 U/mL penicillin, and 100 μg/mL streptomycin) supplemented with 250 U/mL IFNβ (PBL Assay Science), 12.5 ng/mL IFNγ (Peprotech) and 20 ng/mL IL-3. Primed HSPC-pDCs were phenotypically validated using flow cytometry and used for virus inoculation.

### Cell Lines

Calu-3 epithelial lung cancer cells (kindly provided by Laureano de le Vega, Dundee University, Scotland, UK) and human lung adenocarcinoma epithelial A549 cells expressing hACE2 (kindly provided by Brad Rosenberg, Icahn School of Medicine at Mount Sinai, New York, USA) were grown as a monolayer in DMEM10 (Dulbecco's minimal essential medium, DMEM, Life Technologies, supplemented with 10% (v/v) hiFCS, 2 mM L-glutamine, 100 U/mL penicillin, and 100 μg/mL streptomycin. VeroE6 cells expressing TMPRSS2 (VeroE6-hTMPRSS2, kindly provided by Professor Stefan Pöhlmann, University of Göttingen) (37) were grown in DMEM5 (DMEM supplemented with 5% (v/v) hiFCS, 2 mM L-glutamine, 100 U/mL penicillin, and 100 μg/mL streptomycin), supplemented with 10 μg/mL blasticidin (Invivogen) to maintain TMPRSS2 expression. All cells were cultured at 37° C. and 5% Co₂.

### Air-Liquid Interface (ALI) Epithelium Model

ALI cells were generated and cultured as described previously (10, 38). In brief, primary nasal cells were isolated using a nasal brush (Dent-O-Care). Cells were cultured as a monolayer in tissue culture flasks coated with 0.1 mg/ml Bovine type I collagen solution (Sigma-Aldrich). At passage two, cells were seeded at 2-3×10\hat{ }4 cells on 6.5 mm Transwell membranes (Corning) coated with 30 μg/ml Bovine type I collagen solution and cultured in 2× P/S (200 U/ml Pen/Strep DMEM-low glycose (Sigma-Aldrich) mixed 1:1 (v/v) with 2× Monolayer medium (Airway Epithelium Cell Basal Medium, PromoCell, supplemented with 2 packs of Airway Epithelial Cell Growth Medium Supplement, PromoCell, without triiodothyronine+1 mL of 1.5 mg/ml BSA). When cultures reached confluency, Air-liquid interface (ALI) is introduced and medium is changed to ALI medium (Pneumacult ALI medium kit (StemCell)+ALI medium supplement (StemCell)+100 U/mL Pen/strep) supplemented with 0.48 μg/mL of hydrocortisone (StemCell) and 4 μg/mL heparin (StemCell). Cells were allowed to differentiate for at least 21 days, as verified by extensive cilia beating and mucus covering, prior to experiment initiation.

### Flow Cytometric Analysis

Phenotypic validation of HSPC-pDCs and analysis of ACE2 expression was performed using flow cytometry. Briefly, 1-2×10⁵ cells were washed with facs wash (FW, PBS supplemented with 1% hiFCS and 0.05 mM EDTA (ThermoFisher Scientific)) and stained in FW with antibodies either 30 min on ice or 15 min at room temperature in the dark. Cells were then washed three times and fixated using 1% formaldehyde (Avantor, VWR, Denmark). Fluorescent intensity was measured with a NovoCyte 3000 Analyzer equipped with three lasers (405, 488, and 640 nm) and 13 PMT detectors (ACEA Biosciences, Inc). Data were analyzed using DeNovoSoftware FCS express flow research edition version 6. OneComp eBeads Compensation Beads (ThermoFisher scientific) were used to compensate for fluorescent spillover and gates were set using fluorescent minus one (FMO) controls in each individual experiment. Cells were gated using the following strategy; total cells (SSC-H/FSC-H); single cells (FSC-A/FSC-H); viable cells (LIVE/DEAD Fixable Near-IR Dead Cell Stain Kit, ThermoFisher Scientific Cat #L10119); negative for lineage markers CD3, CD14, CD16, CD19, CD20, CD56 (anti-human Lineage cocktail 1—FITC, BD FastImmune Cat #340546) and negative for CD11c (APC mouse anti-human CD11c, clone B-ly6, BD Pharmingen Cat #559877), and subsequently analyzed for the expression of pDC markers CD123 (PE mouse anti-human CD123, clone 6H6, eBioscience Cat #12-1239-42) and CD304 (BV421 anti-human CD304, clone 12C2, BioLegend Cat #354514). In some experiments, cells were stained for ACE2 expression (PerCP mouse anti-human ACE2, clone AC384, Novus Biologicals Cat #NBP2-80038PCP).

### Virus and Propagation

The SARS-CoV-2 strain FR2020 was kindly provided by Professor Georg Kochs (University of Freiburg) and Professor Arvind Patel (University of Glasgow, UK) kindly provided the SARS-CoV-2 alpha variant. Virus was propagated using VeroE6 cells expressing human TMPRSS2 (37). In brief, 4-6×10⁶ cells were seeded in 5 mL medium in a T75 culture flask and infected at 0.05 multiplicity of infection (MOI). One hour after infection, culture medium was increased up to 10 mL and virus propagation continued up to 72 hrs after infection or if a cytopathic effect (CPE) of approximately 70% was visible. To harvest the virus, cell culture supernatant was removed from the flask, centrifuged at 300 g for 5 minutes to remove cell debris, aliquotted and stored at −80° C. The amount of infectious virus in the generated stock was determined using a limiting dilution assay.

### Infection Assays

2×10⁵ HSPC-pDCs were seeded in a 48-well in 100 μL RF10 supplemented with 20 ng/mL IL-3. 100 μL control medium, medium containing SARS-CoV-2 at 1, 0.1, 0.01 MOI, 2.5 μg/ml R837 for TLR7 stimulation (Imiquimod, InvivoGen), 800 ng/mL poly(I:C) for TLR3 stimulation (Poly(I:C) LMW, InvivoGen), 2,5 μg/ml R848 for TLR7 and TLR8 stimulation (Resiquimod, InvivoGen), 50 ng/mL Pam3CSK4 (Invivogen) for TLR1/2 stimulation or 5 ng/mL Pam2CSK4 (Invivogen) for TLR2/6 stimulation was added for 4 hrs after which the culture was topped up with RF10+IL-3 to a final volume of 1 mL. Cells and supernatants were collected at 4 hrs, 24 hrs, 48 hrs, 72 hrs and 96 hrs post virus inoculation. Supernatants were aliquotted and stored at −80° C. until further analysis by ELISA, MSD or limiting dilution assay. Cells were washed with PBS and stored as pellets at −80° C. until further analysis by RT-qPCR. In some experiments, the SARS-CoV-2 envelope (E) protein (ABclonal RP01263) or the SARS-CoV-2 spike (S) protein (ABclonal RP01283LQ) was added to pDCs at a final concentration of 1 μg/mL. The IRAK4 inhibitor (Pf06650833, Sigma-Aldrich PZ0327) was used at a final concentration of 10 uM. VeroE6 cells constitutively produce low level IL-6 independently of SARS-CoV-2 propagation. Thus to discriminate between de novo IL-6 production by pDCs upon SARS-CoV-2 exposure, mock Vero-virus conditions were run in parallel and the IL-6 signal was subtracted from the actual infection samples, to properly determine IL-6 production by pDCs.

1×10⁵ Calu-3 or A459 hACE2 were seeded in a 48-well in 500 μL DMEM10 and the following day, medium was replaced with 200 L HSPC-pDC conditioned medium or 200 L DMEM10. After 18 hrs, cells were inoculated with SARS-CoV-2 at 0.1 MOI, and after 1 hr the cultures were topped up using DMEM10 to a final volume of 1 mL. Supernatants were collected 48 hrs after virus inoculation, aliquoted and stored at −80° C. until the viral titers were quantified using a limiting dilution assay. To generate HSPC-pDC conditioned medium, HSPC-pDCs were inoculated at 1 MOI or left unexposed. After 3 days supernatants were stored at −80° C. until commencement of the protection experiment. To dilute HSPC-pDC conditioned medium, the medium was diluted 3-fold using DMEM10. To test whether type I IFN contributes to the pDC-mediated inhibition of SARS-CoV-2 inhibition, antibodies blocking the type I IFN receptor (mouse anti-human IFN alpha antibody, clone MMHAR-2, PBL Assay Science Cat #21385-1) or isotype control (Ultra-LEAF Purified mouse IgG2a, clone MOPC-173, BioLegend Cat #400264) were added to Calu-3 cells in 50 μL PBS and antibodies neutralizing IFNα (mouse anti-human IFN alpha antibody, clone MMHA-2, PBL Assay Science Cat #21100-2) or isotype control (Purified mouse IgG1, clone MOPC-21, BioLegend Cat #400102) were added to 200 μl HSPC-pDC conditioned medium, 10 minutes prior to addition of conditioned medium to the Calu-3 cells. The final concentration (after topping up the culture volume) of each antibody was 10 μg/mL.

### Limiting Dilution Assay

To determine the amount of infectious virus in cell culture supernatant or generated virus stocks, a limiting dilution assay was performed. 2×10⁴ VeroE6-TMPRRS2 cells were seeded in 50 μL DMEM5 in a 96 well plate. The next day, samples were thawed and 5× diluted, followed by 10-fold serial dilution using DMEM5, and 50 μL of each dilution was added to the cells. Final dilution range covered 10⁻¹ —10⁻¹¹ in quadruplicate for supernatants or octuplicate for virus stocks. Each well was evaluated for cytopathic effect (CPE) by eye using standard microscopy, and the tissue culture infectious dose 50 (TCID₅₀/mL) was calculated using the Reed and Muench method (39). To convert to the mean number of plaque forming units (pfu)/mL, the TCID₅₀/mL was multiplied by factor 0.7 (ATCC—Converting TCID[50] to plaque forming units (PFU)). Additionally, cells were fixed by adding 10% Formalin (Sigma-Aldrich) at a 1:1 (v/v) ratio, stained with crystal violet solution (Sigma-Aldrich) and stored at room temperature.

### Reverse Transcriptase-Quantitative PCR (RT-qPCR)

To determine expression levels of the human IFNa2a, TNFα, CXCL10, IFNL1, GAPDH, ACE2, TMPRSS2 and SARS-CoV-2 N1 gene, RNA was purified from cells using the RNeasy mini kit (Qiagen) according to the manufacturer's instructions with RNA being eluted in 30 L. Subsequently, 100-200 ng of RNA was used as input for cDNA synthesis using the iScript cDNA synthesis kit (Bio-Rad) on an Arktik thermal cycler (Thermo scientific) with program: 5′25° C.; 20′46° C.; 1′95° C.; 4° C. For commercially available Taqman assays (IFNα2a Hs00265051_s1, CXCL10 Hs00171042_ml, GAPDH Hs02758991_g1, ACE2: Hs01085333_ml and TMPRSS2: Hs01122322_ml, ThermoFisher), samples were analyzed in a 10 μL (final volume) reaction mix containing; 5 μL Taqman Fast Advanced Master Mix, 0.5 L Tagman assay, 3.5 μL Nuclease-free water and 1 μL of cDNA. For the SARS-CoV-2 N1 gene qPCR, primers and probe sequences were provided by the CDC and purchased from Eurofins. Samples were analyzed in a final volume of 10 μL, containing 5 μL Taqman fast Advanced Master Mix, 1 μL fw primer (5 pmol/L 2019-nCoV-N1 fw primer—GAC CC AAA ATC AGC GAA AT), 1 μL rev primer (5 pmol/L 2019-nCoV_N1 rev primer—TCT GGT TAC TGC CAG TTG AAT CTG), 1 μL probe (1.25 pmol/L 2019-nCoV_N1 Probe—FAM-ACC CCG CAT TAC GTT TGG TGG ACC-BHQ1), 2 μL Nuclease-free water and 1 μL of cDNA. Analysis was performed on a Lightcycler 480 platform with program: 2′50° C.; 2′95° C.; 40×(1″95° C.; 20″60° C.). Ct values were extracted using the Lightcycler Software.

### Cytokine Quantification Assays

Supernatants were thawed at room temperature or 4° C. and inactivated by adding 1:1 (v/v) 0.4% Triton-X-100 (35). Protein levels were quantified using the Human DuoSet ELISAs for IL-6, IL-8, TNFα, CXCL10 (R&D Systems) or the Human IFN-α pan ELISA kit (Mabtech 3425-1M-6, detecting IFN-α subtypes 1/13, 2, 4, 5, 6, 7, 8, 19, 14, 16 and 17), according to the manufacturer's instructions on a Synergy SynergyHTX multi-mode platereader (BioTek) using the Gen5 version 3.04 program. Protein levels of IFNα2a, IFNβ, IFNγ and IFNλ1 were quantified using the human U-plex Interferon Combo (Meso Scale Discovery K15094K-2), according to the manufacturer's instructions on the MESO QuickPlex SQ 120.

IL-3, IL-6, IL-8, TNFα and CXCL10 protein levels in plasma from SARS-CoV-2 infected individuals were quantified using the V-PLEX Custom Human Cytokine 54-plex kit (Meso Scale Discovery) according to the manufacturer's instructions with overnight incubation of the diluted samples and standards at 4° C. The electrochemiluminescence signal (ECL) was detected by MESO QuickPlex SQ 120 plate reader (MSD) and analyzed with Discovery Workbench Software (v4.0, MSD).

### Generating Genetically Modified HSPC-pDC

HSPC-pDCs were genetically modified as previously described (20). Briefly, sgRNAs directed at MyD88 (5′-GCTGCTCTCAACATGCGAGTG-3′ (SEQ ID NO: 1)) (20), TICAM1 (TRIF #1: 5′-AACACATCGCCCTGCGGGTT-3′ (SEQ ID NO: 2) and TRIF #2: 5′-CTGGCGACCCCTGTCGCGTG-3′ (SEQ ID NO: 3)), DDX58 (RIG-I #1: 5′-GGTGTTGTTTACTAGTGTTG-3′ (SEQ ID NO: 4) and RIG-I #2: 5′-GGCATCCCCAACACCAACCG-3′ (SEQ ID NO: 5)), TLR1 (TLR1 #1 5′-CAACCAGGAATTGGAATACT-3′ (SEQ ID NO: 6) and TLR1 #2 5-CTGATATTCAAATGAGCAAT-3′ (SEQ ID NO: 7)), TLR2 (TLR2 #1 5′-CTAAATGTTCAAGACTGCCC-3′ (SEQ ID NO: 8) and TLR2 #2 5′-AATCCTGAGAGTGGGAAATA-3′ (SEQ ID NO: 9)), TLR3 (TLR3 #1: 5′-GTACCTGAGTCAACTTCAGG-3′ (SEQ ID NO: 10) and TLR3 #2: 5′-CTGGCTATACCTTGTGAAGT-3′ (SEQ ID NO: 11)), TLR6 (TLR6 #1 5′-TTCCAACTATTATGATCATA-3′ (SEQ ID NO: 12) and TLR6 #2 5′-CAAGTAGCTGGATTCTGTTA-3′ (SEQ ID NO: 13)), TLR7 (TLR7 #1: 5′-CTGTGCAGTCCACGATCACA-3′ (SEQ ID NO: 14) and TLR7 #2: 5′-TCCAGTCTGTGAAAGGACGC-3′ (SEQ ID NO: 15)), TLR8 (TLR8 #1 5′-GTGCAGCAATCGTCGACTAC-3′ (SEQ ID NO: 16) and TLR8 #2 5′-TCCGTTCTGGTGCTGTACAT-3′ (SEQ ID NO: 17)); CD304 (CD304 #1 5′-CCCGGGTACCTTACATCTCC-3′ (SEQ ID NO: 18) and CD304 #2 5′-CTGTCCTCCAAATCGAAGTG-3′ (SEQ ID NO: 19)) and AAVS1 (control sgRNA, 5′-GGGGCCACTAGGGACAGGAT-3′ (SEQ ID NO: 20)) (20) were synthesized by Synthego with the three terminal nucleotides in both ends chemically modified with 2′-O-methyl-3′-phosphorothioate. Thawed CD34⁺ HSPCs were cultured at low density (10⁵ cells/mL) for 3-4 days in SFEM II medium supplemented with 20 U/mL penicillin and 20 μg/mL streptomycin, 100 ng/mL Flt3-L, 50 ng/mL TPO, 100 ng/mL SCF, and 35 nM UM171 (STEMCELL technologies). Ribonucleoprotein (RNP) complexes were made by incubating 6 μg Cas9 protein (Alt-R S.p.Cas9 Nuclease V3, Integrated DNA Technologies) with 3.2 μg sgRNA in a final volume of 2 μL at room temperature for 15-20 minutes. 200.000-800.000 HSPCs were washed with PBS, resuspended in 20 μL 50 mM Mannitol buffer (made in house; 5 mM KCl, 120 mM Na₂HPO₄/NaH₂PO₄, pH 7.2, 15 mM MgCl₂), added to the RNP complexes and transferred to a Nucleocuvette strip chamber (Lonza). In case of multiple sgRNAs were used for nucleoporation (i.e. TRIF, RIG-I and double knockouts), individual sgRNAs were incubated with Cas9 protein, after which they were pooled and added to the cells. Nucleoporation was performed using the Lonza 4D-Nucleofector™ System (program DZ100) and HSPC were subsequently cultured for 21 days in HSPC-pDC differentiation medium as described above. CRISPR-Cas9 induced genetic modification were validated at the genomic and protein level, as described below.

### DNA Extraction, PCR and Sequencing.

To validate the CRISPR-Cas9 induced genetic modification at the genetic level, cells were harvested and DNA was extracted using the DNeasy Blood & Tissue Kit (Qiagen Cat #69504). Amplicons were generated using 100 ng DNA as input in a final volume of 40 μL (consisting of 8 μl 5× Phusion GC buffer (Phusion High-Fidelity DNA Polymerase set (ThermoFisher Scientific, Cat #F534), 0.8 μL dNTPs (dNTP Set, 100 mM, InvivoGen Cat #10297117), 0,4 μL Phusion Green High-Fidelity DNA polymerase (ThermoFisher Scientific), 2 μL (10 μM) fw primer, 2 μL (10 μM) rev primer and nuclease free water) on an Arktik Thermal Cycler (ThermoFisher Scientific) with program: 1′98° C.; 35×(10″98° C.; 30″68° C.; 1′72° C.; 10′72° C.; 4° C.). The following primers were used: MyD88 fw: 5′-CTC CGT GGA AGA ACT GTG GC-3′ (SEQ ID NO: 21); MyD88 rev: 5′-GGC GGC TGT ATC CAA CGC-3′ (SEQ ID NO: 22); AAVS1 fw: 5′-TCA GTG AAA CGC ACC AGA CA-3′ (SEQ ID NO: 23); AAVS1 rev 5′-CCA CTA CTA CGC CTG GAT GT-3′ (SEQ ID NO: 24); TRIF fw: 5′-AAA CCA GCA CCA ACT ACC CA-3′ (SEQ ID NO: 25); TRIF rev #1: 5′-TAG GCT GAG TAG GCT GCG TT-3′ (SEQ ID NO: 26); TRIF rev #2: 5′-CCC CCA AAG GGC ATT CGA G-3′ (SEQ ID NO: 27); RIG-I fw #1: 5′-CTA AGG ACT TGC CTA CAG CT-3′ (SEQ ID NO: 28); RIG-I fw #2 5′-GGC TCT GTG CTA AGG ACT TG-3′ (SEQ ID NO: 29); RIG-I rev #1: 5-TGC TTG GGA TGA GAG CTC AG-3′ (SEQ ID NO: 30); RIG-I rev #2: 5′-CAG ATA GCC AAG AGC TGG GC-3′ (SEQ ID NO: 31); TLR1 fw: 5′-TGG TGA GCC ACC ATT CAA CC-3′ (SEQ ID NO: 32); TLR1 rev: 5′-TGC GTG TAC CAG ACA CTG TG-3′ (SEQ ID NO: 33); TLR2 fw: 5′-CTT GCT CTG TAA TTCC GGA TGG-3′ (SEQ ID NO: 34); TLR2 rev: 5′-TGC AGC CTC CGG ATT GTT AAC-3′ (SEQ ID NO: 35); TLR3 fw: 5′-AGC TGC AAC TGG CAT TAG GGT G-3′ (SEQ ID NO: 36); TLR3 rev: 5′-GGG AGA AAG CGA GAG AGG CA-3′ (SEQ ID NO: 37); TLR6 fw: 5′-GCC TAT ATT GCC CCT TCT GGC-3′ (SEQ ID NO: 38); TLR6 rev: 5′-CCA CAG GTT TGG GCC AAA GA-3′ (SEQ ID NO: 39); TLR7 fw: 5′-ATG CTG CTT CTA CCC TCT CGA-3′ (SEQ ID NO: 40); TLR7 rev: 5′—AGT AGG GAC GGC TGT GAC AT-3′ (SEQ ID NO: 41); TLR8 fw: 5′-TTG GGA TTA CAG GTG TGA GCC-3′ (SEQ ID NO: 42); TLR8 rev: 5′-TTG GGA TTA CAG GTG AGC C-3′ (SEQ ID NO: 43). Amplicons were separated on a 1% agarose gel using FastDigest Green Buffer (10×, ThermoFisher Scientific, Cat #B72), appropriate bands were excised and purified using the E.Z.N.A Extraction Kit (Omega Bio-Tek, Cat #D2500-01), according to manufactures' instructions. Isolated amplicons (60 ng) were sent for sequencing with 2.5 μM of a single primer in a total volume of 10 μL to Eurofins Genomics. Sequences were subsequently analyzed using the Interference of CRISPR Edits online tool (ICE, Synthego). In addition to validating the mutations, all control samples were validated to have an intact sequence spanning 300 bp up and downstream the targeted region.

### Western Blot Analysis.

Cells were washed with ice cold PBS and lysed in (200.000 cells/100 μL) Ripa buffer (Thermofisher Scientific) supplemented with Pierce protease and phosphatase inhibitors (Thermofisher Scientific, A32961), Complete Ultra protease inhibitor (Roche, 05892791001), Sodium fluoride (Avantar) and Benzonase Nuclease (Sigma-Aldrich), for 15 minutes on ice and stored at −20° C. Samples were thawed on ice, diluted 1:1 (v/v) with Laemmli sample buffer (Sigma-Aldrich, S3401), incubated at 95° C. for 4 min, cooled on ice for 5 min, 30 μL was loaded for MyD88 and RIG-I analysis and 40 μL for TRIF analysis, together with Precision Plus Protein Kaleidoscope protein marker (Bio-Rad 1610395) onto a 10% Criterion TGX Precast Midi Protein Gel (18 well Bio-Rad, 5671034) in Nu PAGE MOPS SDS running buffer (Thermo Scientific NP0001). Proteins were transferred onto a Trans-Blot Turbo Midi PVDF Transfer membrane (Bio-Rad, 170-4157) using the Trans-Blot Turbo Transfer System (Bio-Rad). Membranes were washed using Tris-buffered saline (Fisher Scientific) supplemented with 0.05% (v/v) Tween 20 (Sigma-Aldrich) (TBS-T), blocked for 1 hr at room temperature (RT) in 5% Skim Milk Powder (Sigma-Aldrich) in TBS-T, washed with TBS-T, incubated over-night (o/n) at 4° C. with primary antibody diluted 1:500 in 5% Bovine Serum Albumin Fraction V (Roche 10735086001) in TBS-T. The following morning, membranes were washed with TBS-T, incubated for 1 hr with secondary antibody diluted 1:7500 in 5% Skim Milk, washed, and proteins were visualized using Clarity Western ECL Substrate (Bio-Rad, 170560) for MyD88 analysis and SuperSignal West Femto Maximum Sensitivity Substrate (ThermoFisher Scientific, 34095) for RIG-I and TRIF analysis on an ImageQuant LAS 4000 mini biomolecular imager (GE Healthcare). Membranes were washed with TBS-T, and incubated o/n at 4° C. with the primary antibody diluted 1:10.000 for the loading control. To detect MyD88, rabbit-anti-human (r-a-h)MyD88 (clone D80F5, 33 kDa, Cell Signaling Technology, cat #4283) was used. For TRIF, r-a-hTRIF (98 kDa, Cell Signaling Technology, cat #4596) was used, and for RIG-I, r-a-hRIG-I (clone D14G6, 102 kDa, Cell Signaling Technology, cat #3743) was used. Each membrane was re-used to for the loading control vinculin (mouse-a-hVCL, clone hVIN-1, 116 kDa, Sigma-Aldrich, cat #V9131). As secondary antibodies, peroxidase-conjugated donkey-anti-rabbit and donkey-anti-mouse was used (Jackson Immuno Research 711-036-152 and 715-036-150).

NanoString nCounter Analyses

To perform broad transcriptomic profiling on SARS-CoV-2 exposed HSPC-pDCs, an nCounter NanoString analysis was performed (NanoString Technologies, Seattle, WA, USA). HSPC-pDCs from two donors were exposed to SARS-CoV-2 at 1 MOI (for 4, 24 or 48 hrs and mock treated samples at 4 and 48 hrs), after which cell pellets were collected and RNA extracted with the RNeasy mini kit (Qiagen). 30 ng of RNA was used as input for the analysis using the nCounter SPRINT Profiler (NanoString Technologies) and the nCounter PanCancer Immune Profiling Panel (cat #XT-CSO-HIP1-12) plus a custom made PanelPlus of the following genes: NFE2L2, TMEM173, MB21D1, IFNLR1, IRF9, IFNL3, IFNL4, AIM2, TREX1, ENPP1, PCBP1, PQBP1, G3BP1, STIM1, LRRC8A, SLC19A1, NLRC3, NLRX1, ZDHHC1, TRIM56, TRIM32, RNF5, ULK1, TTLL4, TTLL6, AGBL5, AGBL4, PRKDC, DDX41. Analysis was performed according to the manufacturer's protocol using a 20 hours hybridization time.

The raw data were processed using the nSOLVER 4.0 software (NanoString Technologies) for D^{high} and D^{low} separately to ensure proper normalization of each dataset. Firstly, a positive control normalization was performed using the geometric mean of all positive controls except for the control named F, as recommended by the manufacturer. Finally, a second normalization was performed using the geometric mean of housekeeping genes with reasonable expression levels and low coefficient of variance percentage (ABCF1, AMMECR1L, CNOT10, CNOT4, DDX50, EDC3, POLR2A, TBP, TLK2 and ZNF143 for D^{high} and G6PD, GPATCH3, MRPS5, MTMR14, POLR2A and SDHA for D^{low}), before exporting the data to Excel (Microsoft Corporation, Redmond, WA, USA). Background threshold levels were calculated based on the mean plus two standard deviations of the eight negative controls. Genes with an average expression below the threshold were excluded from further analyses. Data were plotted using Prism 8.2.0 (GraphPad, La Jolla, CA, USA) and R software version 3.5.1 with the following packages installed: ggplot2, circlize, dendextend, ComplexHeatmap and RColorBrewer.

### Reactome Pathway Overrepresentation Analysis

To assign pathways to the gene clusters identified in pDCs from D^{high} and D^{low} 48 hrs after SARS-CoV-2 exposure using unsupervised hierarchical cluster analysis on the NanoString nCounter data, we utilized the Reactome Pathway Browser version 3.7, database release 75 (1tt s://reactome.org/PathwayBrowser); a comprehensive web-based resource for curated human pathways. Disease pathways were excluded from the analyses and we used UniProt as the source of entities (maximum pathway size was 400). Only six genes were not assigned to any pathways in Reactome. Reactome defines statistically significantly enriched pathways using a Binomial Test, followed by correction for multiple comparisons by the Benjamini-Hochberg approach (40).

### Statistical Analysis

Differences between experimental conditions were analyzed using the ratio paired student T test with GraphPad Prism (Version 6). P-values<0.05 were considered significant: *p<0.05, **p<0.01, ***p<0.001, ****p<0.0001. To determine correlation between IFNα production by pDCs and time of exposure to SARS-CoV-2, as well as to compare gene expression changes in D^{high} and D^{low} after 4 and 48 hrs after exposure to SARS-CoV-2, simple linear regression analysis were performed using GraphPad Prism. The R squared and p-value are indicated in the figures.

### REFERENCES

1. Q. Zhang et al., Inborn errors of type I IFN immunity in patients with life-threatening COVID-19. Science 370, (2020).
2. A. Banerjee, A. Berezhkovskii, R. Nossal, Kinetics of cellular uptake of viruses and nanoparticles via clathrin-mediated endocytosis. Phys Biol 13, 016005 (2016).
3. N. Zhu et al., A Novel Coronavirus from Patients with Pneumonia in China, 2019. N Engl J Med 382, 727-733 (2020).
4. F. Wu et al., A new coronavirus associated with human respiratory disease in China. Nature 579, 265-269 (2020).
5. P. Brodin, Immune determinants of COVID-19 disease presentation and severity. Nat Med 27, 28-33 (2021).
6. B. Hu, H. Guo, P. Zhou, Z. L. Shi, Characteristics of SARS-CoV-2 and COVID-19. Nat Rev Microbiol, 1-14 (2020).
7. D. Blanco-Melo et al., Imbalanced Host Response to SARS-CoV-2 Drives Development of COVID-19. Cell 181, 1036-1045 e1039 (2020).
8. J. Hadjadj et al., Impaired type I interferon activity and inflammatory responses in severe COVID-19 patients. Science 369, 718-724 (2020).
9. X. Zhang et al., Viral and host factors related to the clinical outcome of COVID-19. Nature 583, 437-440 (2020).
10. P. Bastard et al., Autoantibodies against type I IFNs in patients with life-threatening COVID-19. Science 370, (2020).
11. L. Dalskov et al., SARS-CoV-2 evades immune detection in alveolar macrophages. EMBO Rep, e51252 (2020).
12. M. A. Niles et al., Macrophages and Dendritic Cells Are Not the Major Source of Pro-Inflammatory Cytokines Upon SARS-CoV-2 Infection. Front Immunol 12, 647824 (2021).
13. X. Yin et al., MDA5 Governs the Innate Immune Response to SARS-CoV-2 in Lung Epithelial Cells. Cell Rep 34, 108628 (2021).
14. A. Wahl et al., SARS-CoV-2 infection is effectively treated and prevented by EIDD-2801. Nature, (2021).
15. M. Swiecki, M. Colonna, The multifaceted biology of plasmacytoid dendritic cells. Nat Rev Immunol 15, 471-485 (2015).
16. M. Liao et al., Single-cell landscape of bronchoalveolar immune cells in patients with COVID-19. Nat Med 26, 842-844 (2020).
17. R. Zhou et al., Acute SARS-CoV-2 Infection Impairs Dendritic Cell and T Cell Responses. Immunity 53, 864-877 e865 (2020).
18. A. Benard et al., Interleukin-3 is a predictive marker for severity and outcome during SARS-CoV-2 infections. Nat Commun 12, 1112 (2021).
19. Y. Kasuga, B. Zhu, K. J. Jang, J. S. Yoo, Innate immune sensing of coronavirus and viral evasion strategies. Exp Mol Med 53, 723-736 (2021).
20. A. Laustsen et al., Interferon priming is essential for human CD34⁺ cell-derived plasmacytoid dendritic cell maturation and function. Nat Commun 9, 3525 (2018).
21. F. Onodi et al., SARS-CoV-2 induces human plasmacytoid predendritic cell diversification via UNC93B and IRAK4. JExp Med 218, e20201387 (2021).
22. G. K. Geiss et al., Direct multiplexed measurement of gene expression with color-coded probe pairs. Nat Biotechnol 26, 317-325 (2008).
23. A. Szabo et al., TLR ligands upregulate RIG-I expression in human plasmacytoid dendritic cells in a type I IFN-independent manner. Immunol Cell Biol 92, 671-678 (2014).
24. S. Torres et al., Differential expression of Toll-like receptors in dendritic cells of patients with dengue during early and late acute phases of the disease. PLoS Negl Trop Dis 7, e2060 (2013).
25. M. Yoneyama, T. Fujita, Structural mechanism of RNA recognition by the RIG-I-like receptors. Immunity 29, 178-181 (2008).
26. M. Tatematsu, F. Nishikawa, T. Seya, M. Matsumoto, Toll-like receptor 3 recognizes incomplete stem structures in single-stranded viral RNA. Nat Commun 4, 1833-1846 (2013).
27. T. Liu et al., The role of interleukin-6 in monitoring severe case of coronavirus disease 2019. EMBO Mol Med 12, e12421 (2020).
28. J. Zhang et al., Serum interleukin-6 is an indicator for severity in 901 patients with SARS-CoV-2 infection: a cohort study. J Transl Med 18, 406-414 (2020).
29. M. Zheng et al., TLR2 senses the SARS-CoV-2 envelope protein to produce inflammatory cytokines. Nat Immunol 22, 829-838 (2021).
30. L. Oliveira-Nascimento, P. Massari, L. M. Wetzler, The Role of TLR2 in Infection and Immunity. Front Immunol 3, 79 (2012).
31. L. Cantuti-Castelvetri et al., Neuropilin-1 facilitates SARS-CoV-2 cell entry and infectivity. Science 370, 856-860 (2020).
32. J. L. Daly et al., Neuropilin-1 is a host factor for SARS-CoV-2 infection. Science 370, 861-865 (2020).
33. S. L. Fanning et al., Receptor cross-linking on human plasmacytoid dendritic cells leads to the regulation of IFN-alpha production. J Immunol 177, 5829-5839 (2006).
34. E. Grage-Griebenow et al., Anti-BDCA-4 (neuropilin-1) antibody can suppress virus-induced IFN-alpha production of plasmacytoid dendritic cells. Immunol Cell Biol 85, 383-390 (2007).
35. U. Hasan et al., Human TLR10 is a functional receptor, expressed by B cells and plasmacytoid dendritic cells, which activates gene transcription through MyD88. J Immunol 174, 2942-2950 (2005).
36. S. N. Lester, K. Li, Toll-like receptors in antiviral innate immunity. J Mol Biol 426, 1246-1264 (2014).
37. M. Hoffmann, H. Kleine-Weber, S. Schroeder, N. Kruger, T. Herrler, S. Erichsen, T. S. Schiergens, G. Herrler, N. H. Wu, A. Nitsche, M. A. Muller, C. Drosten, S. Pohlmann, SARS-CoV-2 Cell Entry Depends on ACE2 and TMPRSS2 and Is Blocked by a Clinically Proven Protease Inhibitor. Cell 181, 271-280 e278 (2020).
38. D. Olagnier, E. Farahani, J. Thyrsted, J. Blay-Cadanet, A. Herengt, M. Idorn, A. Hait, B. Hernaez, A. Knudsen, M. B. Iversen, M. Schilling, S. E. Jorgensen, M. Thomsen, L. S. Reinert, M. Lappe, H. D. Hoang, V. H. Gilchrist, A. L. Hansen, R. Ottosen, C. G. Nielsen, C. Moller, D. van der Horst, S. Peri, S. Balachandran, J. Huang, M. Jakobsen, E. B. Svenningsen, T. B. Poulsen, L. Bartsch, A. L. Thielke, Y. Luo, T. Alain, J. Rehwinkel, A. Alcami, J. Hiscott, T. H. Mogensen, S. R. Paludan, C. K. Holm, SARS-CoV2-mediated suppression of NRF2-signaling reveals potent antiviral and anti-inflammatory activity of 4-octyl-itaconate and dimethyl fumarate. Nat Commun 11, 4938 (2020).
39. L. J. M. Reed, H., A simple method of estimating fifty percent endpoints. The American Journal of Hygiene 27, 493-497 (1938).
40. B. Jassal, L. Matthews, G. Viteri, C. Gong, P. Lorente, A. Fabregat, K. Sidiropoulos, J. Cook, M. Gillespie, R. Haw, F. Loney, B. May, M. Milacic, K. Rothfels, C. Sevilla, V. Shamovsky, S. Shorser, T. Varusai, J. Weiser, G. Wu, L. Stein, H. Hermjakob, P. D′Eustachio, The reactome pathway knowledgebase. Nucleic Acids Res 48, D498-D503 (2020).

### Example 2

Following the general method as set out in “Generating genetically modified HSPC-pDC” for Example 1 above, Cas9 ribonucleoprotein (RNP) complexes consisting of Cas9 protein complexed to one or more (e.g. two) IL-6 ORF-targeting sgRNA are delivered by electroporation into HSPCs prior to differentiation into pDCs, or into the pDCs after differentiation. IL-6 ORF-targeting sgRNAs are selected based on prediction algorithms that identify PAM-containing sites in the genome (for Streptococcus pyogenes Cas9 this would be the sequence 5′-NGG-3′). The next criteria for sgRNA selection would be sgRNAs with minimal target site homology to other sites in the genome to ensure high specificity. Upon introduction of Cas9 RNP, insertion or deletions (INDELs) would be created at the target site in the IL-6 ORF, and should preferably exceed >90% of alleles as analyzed by DNA sequencing methods. To increase efficiencies, dual sgRNAs can be used that lead to deletion of the intervening sequence between the two sites. Following electroporation, pDC differentiation is conducted as in Example 1. The result is a population of pDCs of which the majority of cells (expectedly>80%) carry inactivating mutations in the IL-6 gene and therefore does not express IL-6 but retains the ability to produce type I and III IFNs in response to virus. A minority of the cells are expected to carry no inactivating mutations in the IL-6 ORF and would retain the ability to express IL-6. Another minority of the cells would be expected to carry inactivating mutations only at one IL-6 allele, and these cells would have reduced capacity to express IL-6.

### Example 3

pDCs produced in accordance with Example 1 and Example 2 are tested for cytokine responses to virus and/or the ability to reduce viral infection following the same experimental protocols as in Example 1. The pDCs may be administered to samples together with a general anti-inflammatory (e.g. dexamethasone) and/or a targeted anti-inflammatory (e.g. IL-6 antigonist such as tocilizumab), which is administered either simultaneously with the pDCs, or after a suitable time interval. Enhanced anti-viral activity and/or reduced undesirable inflammation will be observed.

Unless indicated otherwise, the methods used are standard biochemistry and molecular biology techniques.

1. A method of treating or preventing a disease in a subject comprising administering a plasmacytoid dendritic cell (pDC), or a composition comprising said cell, to the subject, wherein the disease is a viral infection, or a disease or complication associated with a viral infection.

2. The method of claim 1, wherein:
(a) the method additionally comprises administration of an anti-viral and/or anti-inflammatory agent;
(b) the pDC is unable to express IL-6 or exhibits reduced IL-6 expression; and/or
(c) the pDC exhibits reduced or no CD304 expression.

3. The method of claim 1 or 2, wherein said pDC is engineered by transformation with an exogenous construct which prevents or reduces IL-6 expression by the pDC.

4. The method of claim 3, wherein IL-6 expression by the pDC is prevented or reduced by deletion or disruption of TLR2.

5. The method of any one of the preceding claims, wherein the pDC is engineered by transformation with an exogenous construct which deletes or disrupts CD304.

6. The method of any one of the preceding claims, wherein the anti-viral and/or anti-inflammatory agent is administered simultaneously with the pDC, or at a suitable time interval subsequent to administration of the pDC; optionally wherein the time interval is at least around 4 hours, 8 hours, 12 hours, or 24 hours.

7. The method of any one of the preceding claims, wherein the anti-inflammatory agent is:
(a) An IL-6 antagonist; optionally comprising an antibody specific for IL-6 or IL-6R, such as tocilizumab, sarilumab, siltuximab, sirukumab, clazakizumab, elsilimomab, or levilimab;
(b) A corticosteroid, such as dexamethasone, hydrocortisone or methylprednisolone;
(c) A non-steroidal anti-inflammatory drug (NSAID), such as ibuprofen.

8. The method of any one of the preceding claims, wherein the virus is a coronavirus or influenza virus, optionally SARS-CoV-2, SARS-CoV or MERS-CoV, optionally wherein the disease is COVID-19.

9. The method of any one of the preceding claims, wherein the pDC is a stem cell-derived pDC.

10. The method of any one of the preceding claims, wherein the pDC expresses IFN type I and/or IFN type III cytokines.

11. A pDC, or a composition comprising said cell, as defined in any one of the preceding claims, optionally for use in a method of treating or preventing a viral infection or a disease or complication associated with a viral infection.

12. A pharmaceutical composition comprising the pDC of claim 11 and a pharmaceutically acceptable preservative, diluent, excipient, or carrier.

13. The composition of claim 11 or 12, additional comprising an anti-inflammatory agent, optionally an anti-inflammatory agent as defined in claim 7.

14. A method of generating a composition comprising a pDC comprising:
(a) providing hematopoietic stem progenitor cells (HSPCs);
(b) incubating said HSPCs in one or more media comprising cytokines, growth factor and/or interferons (IFNs), whereby said HSPCs are differentiated into precursor-pDCs and into pDCs incubating said HSPCs in one or more media, which media may comprise one or more cytokines, growth factors, interferons (IFNs) and/or aryl hydrocarbon receptor (1) antagonists (such as stemregenin-1), whereby said HSPCs are differentiated into precursor-pDCs and into pDCs; and
(c) transforming said HSPCs prior to differentiation, or transforming said pDCs subsequent to differentiation, with an exogenous construct which prevents or reduces IL-6 expression or which prevents or reduces CD304 expression.

15. The method of claim 14, which further comprises formulating the pDC with a pharmaceutically acceptable preservative, diluent, excipient, or carrier.

16. The method of any one of claims 14 or 15, wherein the HSPCs are harvested from a sample obtained from a subject in need of treatment for or prevention of a disease, wherein the disease is a viral infection, or a disease or complication associated with a viral infection.


SEQUENCE LISTING (43 sequences)

SEQ ID NO: 1 - RNA, 21 nt, synthetic construct
  source 1..21: mol_type = other RNA
gctgctctcaacatgcgagtg

SEQ ID NO: 2 - RNA, 20 nt, synthetic construct
  source 1..20: mol_type = other RNA
aacacatcgccctgcgggtt

SEQ ID NO: 3 - RNA, 20 nt, synthetic construct
  source 1..20: mol_type = other RNA
ctggcgacccctgtcgcgtg

SEQ ID NO: 4 - RNA, 20 nt, synthetic construct
  source 1..20: mol_type = other RNA
ggtgttgtttactagtgttg

SEQ ID NO: 5 - RNA, 20 nt, synthetic construct
  source 1..20: mol_type = other RNA
ggcatccccaacaccaaccg

SEQ ID NO: 6 - RNA, 20 nt, synthetic construct
  source 1..20: mol_type = other RNA
caaccaggaattggaatact

SEQ ID NO: 7 - RNA, 20 nt, synthetic construct
  source 1..20: mol_type = other RNA
ctgatattcaaatgagcaat

SEQ ID NO: 8 - RNA, 20 nt, synthetic construct
  source 1..20: mol_type = other RNA
ctaaatgttcaagactgccc

SEQ ID NO: 9 - RNA, 20 nt, synthetic construct
  source 1..20: mol_type = other RNA
aatcctgagagtgggaaata

SEQ ID NO: 10 - RNA, 20 nt, synthetic construct
  source 1..20: mol_type = other RNA
gtacctgagtcaacttcagg

SEQ ID NO: 11 - RNA, 20 nt, synthetic construct
  source 1..20: mol_type = other RNA
ctggctataccttgtgaagt

SEQ ID NO: 12 - RNA, 20 nt, synthetic construct
  source 1..20: mol_type = other RNA
ttccaactattatgatcata

SEQ ID NO: 13 - RNA, 20 nt, synthetic construct
  source 1..20: mol_type = other RNA
caagtagctggattctgtta

SEQ ID NO: 14 - RNA, 20 nt, synthetic construct
  source 1..20: mol_type = other RNA
ctgtgcagtccacgatcaca

SEQ ID NO: 15 - RNA, 20 nt, synthetic construct
  source 1..20: mol_type = other RNA
tccagtctgtgaaaggacgc

SEQ ID NO: 16 - RNA, 20 nt, synthetic construct
  source 1..20: mol_type = other RNA
gtgcagcaatcgtcgactac

SEQ ID NO: 17 - RNA, 20 nt, synthetic construct
  source 1..20: mol_type = other RNA
tccgttctggtgctgtacat

SEQ ID NO: 18 - RNA, 20 nt, synthetic construct
  source 1..20: mol_type = other RNA
cccgggtaccttacatctcc

SEQ ID NO: 19 - RNA, 20 nt, synthetic construct
  source 1..20: mol_type = other RNA
ctgtcctccaaatcgaagtg

SEQ ID NO: 20 - RNA, 20 nt, synthetic construct
  source 1..20: mol_type = other RNA
ggggccactagggacaggat

SEQ ID NO: 21 - DNA, 20 bp, synthetic construct
  source 1..20: mol_type = other DNA
ctccgtggaagaactgtggc

SEQ ID NO: 22 - DNA, 18 bp, synthetic construct
  source 1..18: mol_type = other DNA
ggcggctgtatccaacgc

SEQ ID NO: 23 - DNA, 20 bp, synthetic construct
  source 1..20: mol_type = other DNA
tcagtgaaacgcaccagaca

SEQ ID NO: 24 - DNA, 20 bp, synthetic construct
  source 1..20: mol_type = other DNA
ccactactacgcctggatgt

SEQ ID NO: 25 - DNA, 20 bp, synthetic construct
  source 1..20: mol_type = other DNA
aaaccagcaccaactaccca

SEQ ID NO: 26 - DNA, 20 bp, synthetic construct
  source 1..20: mol_type = other DNA
taggctgagtaggctgcgtt

SEQ ID NO: 27 - DNA, 18 bp, synthetic construct
  source 1..18: mol_type = other DNA
cccccaaagggcattcga

SEQ ID NO: 28 - DNA, 20 bp, synthetic construct
  source 1..20: mol_type = other DNA
ctaaggacttgcctacagct

SEQ ID NO: 29 - DNA, 20 bp, synthetic construct
  source 1..20: mol_type = other DNA
ggctctgtgctaaggacttg

SEQ ID NO: 30 - DNA, 20 bp, synthetic construct
  source 1..20: mol_type = other DNA
tgcttgggatgagagctcag

SEQ ID NO: 31 - DNA, 20 bp, synthetic construct
  source 1..20: mol_type = other DNA
cagatagccaagagctgggc

SEQ ID NO: 32 - DNA, 20 bp, synthetic construct
  source 1..20: mol_type = other DNA
tggtgagccaccattcaacc

SEQ ID NO: 33 - DNA, 20 bp, synthetic construct
  source 1..20: mol_type = other DNA
tgcgtgtaccagacactgtg

SEQ ID NO: 34 - DNA, 22 bp, synthetic construct
  source 1..22: mol_type = other DNA
cttgctctgtaattccggatgg

SEQ ID NO: 35 - DNA, 21 bp, synthetic construct
  source 1..21: mol_type = other DNA
tgcagcctccggattgttaac

SEQ ID NO: 36 - DNA, 22 bp, synthetic construct
  source 1..22: mol_type = other DNA
agctgcaactggcattagggtg

SEQ ID NO: 37 - DNA, 20 bp, synthetic construct
  source 1..20: mol_type = other DNA
gggagaaagcgagagaggca

SEQ ID NO: 38 - DNA, 21 bp, synthetic construct
  source 1..21: mol_type = other DNA
gcctatattgccccttctggc

SEQ ID NO: 39 - DNA, 20 bp, synthetic construct
  source 1..20: mol_type = other DNA
ccacaggtttgggccaaaga

SEQ ID NO: 40 - DNA, 21 bp, synthetic construct
  source 1..21: mol_type = other DNA
atgctgcttctaccctctcga

SEQ ID NO: 41 - DNA, 20 bp, synthetic construct
  source 1..20: mol_type = other DNA
agtagggacggctgtgacat

SEQ ID NO: 42 - DNA, 21 bp, synthetic construct
  source 1..21: mol_type = other DNA
ttgggattacaggtgtgagcc

SEQ ID NO: 43 - DNA, 19 bp, synthetic construct
  source 1..19: mol_type = other DNA
ttgggattacaggtgagcc